SEQUENCE LISTING (4 sequences)

SEQ ID NO: 1 - protein, 483 aa, Bacillus sp. AA560
HHNGTNGTMMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVKAVEVNPNNRNQEVSGEYTIEAWTKFDFPGRGNTHSNFKWRWYHFDGVDWDQSRKLNNRIYKFRGKGWDWEVDTEFGNYDYLMYADIDMDHPEVVNELRNWGVWYTNTLGLDGFRIDAVKHIKYSFTRDWINHVRSATGKNMFAVAEFWKNDLGAIENYLNKTNWNHSVFDVPLHYNLYNASKSGGNYDMRQIFNGTVVQKHPMHAVTFVDNHDSQPEEALESFVEEWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNKAGTVTINADGWGNFSVNGGSVSIWVNK

SEQ ID NO: 2 - protein, 299 aa, Bacillus Bogoriensis
ANSGFYVSGTTLYDANGNPFVMRGINHGHAWYKDQATTAIEGIANTGANTVRIVLSDGGQWTKDDIHTVRNLISLAEDNHLVAVLEVHDATGYDSIASLNRAVDYWIEMRSALIGKEDTVIINIANEWFGSWEGDAWADGYKQAIPRLRNAGLNHTLMVDAAGWGQFPQSIHDYGREVFNADPQRNTMFSIHMYEYAGGNASQVRTNIDRVLNQDLALVIGEFGHRHTNGDVDEATIMSYSEQRGVGWLAWSWKGNGPEWEYLDLSNDWAGNNLTAWGNTIVNGPYGLRETSRLSTVFQ

SEQ ID NO: 3 - protein, 399 aa, Bacillus subtilis
ADLGHQTLESNDGWGAYSTGTTGGSKASSSHVYTVSNRNQLVSALGKPTNTTPKIIYIKGTIDFNVDDNLKPLGLNDYKDPEYDLDKYLKAYDPSTWGKKEPSGPLEEARARSQKNQKARVMVDIPANTTIVGSGTNAIIVGGNFHIKSDNVIIRNIEFQDAYDYFPQWDPTDGSSGNWNSQYDNITINGGTHIWIDHCTFNDGSRPDSTSPTYFGRPYQHHDGQTDASNGANYITMSYNYYHDHDKSSIFGSSDSKISDDGKLKITLHHNRYKNIVQRAPRVRFGQVHVYNNYYEGSTSSSDYPFSYAWGIGKSSKIYAQNNVIDVPGLPAAKTIKVFSGGTALYDSGTLLNGTQINASAANGLSSSVGWTPSLHGTIDASAHVKSNVISQAGAGKLN

SEQ ID NO: 4 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

(19) United States
(12) Patent Application Publication
(10) Pub. No.: US 2017/0328875 A1
(43) Pub. Date: Nov. 16, 2017

(54) Assay and Method for Testing

(71) Applicant: Novozymes A/S, Bagsvaerd (DK)

(72) Inventors: Liv Spangner Christensen, Gentofte (DK); Rune Nygaard Monrad, Hillerod (DK); Lorena Gonzalez Palmen, Malmo (SE); Klaus Gori, Copenhagen (DK)

(73) Assignee: Novozymes A/S, Bagsvaerd (DK)

(21) Appl. No.: 15/529,706

(22) PCT Filed: Dec. 16, 2015
(86) PCT No.: PCT/EP2015/079943

§ 371 Date: May 25, 2017

(30) Foreign Application Priority Data
Dec. 16, 2014 (EP) 14198244.7
Dec. 16, 2014 (EP) 14198248.8
May 27, 2015 (EP) 15169413.0

(51) Int. Cl. G01N 31/22; G01N 33/68; G01N 1/30; G01N 33/58; G01N 31/00; G01N 1/00

(52) U.S. Cl. CPC G01N 31/22; G01N 1/30; G01N 33/582; G01N 33/6839; G01N 1/00; G01N 31/00

(57) ABSTRACT

The present invention concerns an assay for testing the presence of a fluorophore, a method for testing the precence of a fluorophore and an item comprising a flurophore.

### REFERENCE TO A SEQUENCE LISTING

This application contains a Sequence Listing in computer readable form, which is incorporated herein by reference.

### Field of the Invention

The present invention concerns an assay and a method for testing the presence of a fluorophore on a first and/or a second surface. The invention further concerns an item with a surface comprising the composition and a composition comprising a fluorophore.

### Description of the Related Art

A fluorophore absorbs light energy of a specific wavelength and re-emits light at a longer wavelength. The wavelengths of the absorbed light, energy transfer efficiency, and time before emission depend on both the fluorophore structure and its chemical environment, as the molecule in its excited state interacts with surrounding molecules. Wavelengths of maximum absorption excitation) and maximum emission (for example, Absorption/Emission=485 nm/517 nm) are the typical terms used to refer to a given fluorophore, but the whole spectrum may be important to consider.

Excitation energies range from ultraviolet through the visible spectrum, and emission energies may continue from visible light into the near infrared region.

Wash performance has traditionally been addressed by use of remission values where an item after wash is submitted to light at 460 nm and the remission of light is measured and can be compared to the remission before wash as described in WO 2002/099091. However, for some stains it is not possible to see the stain by human eye after wash. The item may appear clean after wash, even though the stain or residuals of the stain may still be present on the item. It is therefore very difficult to detect if an item is completely clean or if a part of the stain is still present after wash.

### SUMMARY OF THE INVENTION

The present invention concerns an assay for testing the presence of a fluorophore on a first and/or a second surface comprising the following steps:
a. Preparing a composition comprising a fluorophore having an absorbance wavelength and an emission wavelength,
b. applying the composition to the first surface,
c. submitting the first surface and/or the second surface to a liquid composition,
d. optionally rinsing and/or drying the first surface and/or the second surface,
e. exposing the first surface and/or the second surface to light having a wavelength within +/−30 nm of the absorbance wavelength of the fluorophore, and
f. detecting the light emitted from the fluorophore present on the first surface and/or the second surface.

The invention further concerns a method for testing the presence of a fluorophore on a first and/or a second surface comprising the following steps:
a. Preparing a composition comprising a fluorophore having an absorbance wavelength and an emission wavelength,
b. applying the composition to the first surface,
c. submitting the first surface and/or the second surface to a liquid composition,
d. optionally rinsing and/or drying the first surface and/or the second surface,
e. exposing the first surface and/or the second surface to light having a wavelength within +/−30 nm of the absorbance wavelength of the fluorophore, and
f. detecting the light emitted from the fluorophore present on the first surface and/or the second surface.

The invention also concerns a composition comprising a fluorophore and an item having a surface, which surface comprises a composition comprising a fluorophore.

### Definitions

Absorbance wavelength: By the term “absorbance wavelength” is understood the wavelength, where the fluorophore has its highest absorbtion (corresponding to the peak in the absorption spectrum of the fluorophore).

Composition for cleaning hard surfaces: The term “composition for cleaning hard surfaces” refers to compositions intended for cleaning hard surfaces such as floors, tables, walls, roofs etc. as well as surfaces of hard objects such as cars (car wash) and dishes (dishware).

Dishware: The term “dishware” is intended to mean any form of kitchen utensil used in domestic or industrial kitchen or food industry such as dinner set or tableware such as but not limited to pans, plates, cops, knives, forks, spoons, porcelain etc. The dishware can be made of any suitable material such as metal, glass, rubber, plastic, PVC, acrylics, ceramics, china or porcelain.

Dish washing composition: The term “dish washing composition” refers to compositions comprising detergent components, which composition is intended for cleaning dishes, table ware, glass ware, cutting boards, pots, pans, cutlery and all forms of compositions for cleaning hard surfaces areas in kitchens. The present invention is not restricted to any particular type of dish wash composition or any particular detergent component. The dish washing composition can be used for both domestic dish washing, industrial and institutional dish washing including composition for ADW.

Detergent Composition: The term “detergent composition” refers to compositions that find use in the removal of undesired compounds from surfaces to be cleaned, such as textile surfaces.

The detergent composition may be used to e.g. clean textiles for both household cleaning and industrial cleaning. The terms encompass any materials/compounds selected for the particular type of cleaning composition desired and the form of the product (e.g., liquid, gel, powder, granulate, paste, or spray compositions) and includes, but is not limited to, detergent compositions (e.g., liquid and/or solid laundry detergents and fine fabric detergents; fabric fresheners; fabric softeners; and textile and laundry pre-spotters/pretreatment). The detergent formulation may contain one or more enzymes such as hemicellulases, peroxidases, proteases, cellulases, xylanases, lipases, phospholipases, esterases, cutinases, pectinases, mannanases, pectate lyases, keratinases, reductases, oxidases, phenoloxidases, lipoxygenases, ligninases, pullulanases, tannases, pentosanases, malanases, beta-glucanases, arabinosidases, hyaluronidase, chondroitinase, laccase, DNase, chlorophyllases, amylases, perhydrolases, peroxidases, xanthanase and mixtures thereof. The detergent composition may further comprise detergent ingredients such as surfactants, builders, chelators or chelating agents, bleach system or bleach components, polymers, fabric conditioners, foam boosters, suds suppressors, dyes, perfume, tannish inhibitors, bactericides, fungicides, soil suspending agents, anti-corrosion agents, enzyme inhibitors or stabilizers, enzyme activators, transferase(s), hydrolytic enzymes, oxido reductases, bluing agents and fluorescent dyes, antioxidants, and solubilizers.

Emission wavelength: By the term “emission wavelength” is understood the wavelength, where the fluorophore shows its highest fluorescence emission as a consequence of excitation by absorbtion of light within its absorption area.

Food material: The term “food material” includes any raw material which is to be included in the food product or it may be any intermediate form of the food product which occurs during the production process prior to obtaining the final form of the food product. It may be any individual raw material used and/or any mixture thereof and/or any mixture thereof also including additives and/or processing aids, and/or any subsequently processed form thereof including heat treatment.

The food product may be made from at least one raw material that is of plant origin, for example a vegetable tuber or root, such as but not limited to the group consisting of potato, sweet potato, yams, yam bean, parsnip, parsley root, Jerusalem artichoke, carrot, radish, turnip, and cassava potato; cereal, such as but not limited to the group consisting of wheat, rice, corn, maize, rye, barley, buckwheat, sorghum and oats; coffee; or cocoa. Also included are processed food products like cereal-based dough product such as, e.g., bread, pastry, cake, pretzels, bagels, Dutch honey cake, cookies, gingerbread, gingercake or crispbread or a fried cereal-based dough product, such as, e.g., corn chips, tortilla chips or taco shells.

The food product may be made from at least one raw material that is of animal origin, for example dairy products and meat products. Also food products made from more than one raw material are included in the scope of this invention, for example processed food products like sausages comprising meat, animal fat, polysaccharide, milk ingredients, spices and water.

Hard surface: The term “hard surface” is defined herein as hard surfaces including floors, tables, walls, roofs etc. as well as surfaces of hard objects such as cars (car wash) and dishes (dishware). The hard surface can be in a domestic house, industries or institutions.

Laundering: The term “laundering” relates to both household laundering and industrial laundering and means the process of treating textiles with a liquid composition, e.g. a wash liquor containing a detergent composition. The laundering process can for example be carried out using e.g. a household or an industrial washing machine or can be carried out by hand.

Textile: The term “textile” means any textile material including yarns, yarn intermediates, fibers, non-woven materials, natural materials, synthetic materials, and any other textile material, fabrics made of these materials and products made from fabrics (e.g., garments and other articles). The textile or fabric may be in the form of knits, wovens, denims, non-wovens, felts, yarns, and towelling. The textile may be cellulose based such as natural cellulosics, including cotton, flax/linen, jute, ramie, sisal or coir or manmade cellulosics (e.g. originating from wood pulp) including viscose/rayon, cellulose acetate fibers (tricell), lyocell or blends thereof. The textile or fabric may also be non-cellulose based such as natural polyamides including wool, camel, cashmere, mohair, rabbit and silk or synthetic polymers such as nylon, aramid, polyester, acrylic, polypropylene and spandex/elastane, or blends thereof as well as blends of cellulose based and non-cellulose based fibers. Examples of blends are blends of cotton and/or rayon/viscose with one or more companion material such as wool, synthetic fiber (e.g. polyamide fiber, acrylic fiber, polyester fiber, polyvinyl chloride fiber, polyurethane fiber, polyurea fiber, aramid fiber), and/or cellulose-containing fiber (e.g. rayon/viscose, ramie, flax/linen, jute, cellulose acetate fiber, lyocell). Fabric may be conventional washable laundry, for example stained household laundry. When the term fabric or garment is used it is intended to include the broader term textiles as well.

The composition comprises an amount of fluorophore that allows an enzyme to degrade or partially degrade the composition: The term “the composition comprises an amount of fluorophore that allows an enzyme to degrade or partially degrade the composition” means that the number of fluorophore compounds per number of building block in at least one component of the composition to which the fluorophore is attached allows an enzyme to degrade or partially degrade the component to which the fluorophore is attached.

In one example, the composition comprises a cellulose attached to an amount of fluorophore so that an enzyme can still hydrolyse some of the beta-1,4 linkages in cellulose and thereby degrade the composition. In order for enzymes to show activity on the composition, the amount of fluorophore should be adjusted so the fluorophore attached to the component does not block the enzyme from accessing the component.

Wash liquor: The term “wash liquor” is defined herein as liquid composition, such as a solution or mixture of at least one surfactant and water. The wash liquor can optionally comprise a builder. The wash liquor may comprise a detergent composition for laundry, a dishwashing composition or a detergent composition for cleaning hard surfaces.

Wash performance: Wash performance is expressed as a delta remission value (ΔRem) and can be calculated from Assay IV. One way of calculating the wash performance is by measuring on swatches before and after they are submitted to a liquid composition e.g. a wash liquor. The test swatch to be measured was placed on top of another swatch of same type and colour (twin swatch). With only one swatch of each kind per beaker, a swatch from a replicate wash was used in this way. Remission values for individual swatches were calculated by subtracting the remission value of the swatch before being submitted to the liquid composition from the remission value of the swatch after being submitted to the liquid composition. The total wash performance for each swatch set was calculated as the sum of individual ΔRem.

Calculating an enzyme effect is done by taking the measurements from swatches being submitted to a liquid composition comprising enzymes and subtract with the measurements from swatches being submitted to a liquid composition without enzyme for each swatch. The total enzyme performance is calculated as the sum of individual ΔRem_{enzyme}.

### DETAILED DESCRIPTION OF THE INVENTION

The present invention concerns an assay for testing the presence of a fluorophore on a first and/or a second surface comprising the following steps:
a. Preparing a composition comprising a fluorophore having an absorbance wavelength and an emission wavelength,
a. applying the composition to the first surface,
b. submitting the first surface to a liquid composition, optionally simultaneously with the second surface,
c. optionally rinsing and/or drying the first surface and/or the second surface,
d. exposing the first surface and/or the second surface to light having a wavelength within +/−30 nm of the absorbance wavelength of the fluorophore, and
e. detecting the light emitted from the fluorophore present on the first surface and/or the second surface.

The invention further concerns a method for testing the presence of a fluorophore on a first and/or a second surface comprising the following steps:
a. Preparing a composition comprising a fluorophore having an absorbance wavelength and an emission wavelength,
b. applying the composition to the first surface,
c. submitting the first surface and/or the second surface to a liquid composition,
d. optionally rinsing and/or drying the first surface and/or the second surface,
e. exposing the first surface and/or the second surface to light having a wavelength within +/−30 nm of the absorbance wavelength of the fluorophore,
f. detecting the light emitted from the fluorophore present on the first surface and/or the second surface.

The invention further concerns an item having a surface, which surface comprises a composition comprising a fluorophore.

In addition, the invention concerns a composition comprising a fluorophore.

The advantage of this invention is that it allows testing for the presence of a fluorophore on a surface. The presence of the fluorophore can be tested on a first and/or a second surface after the surface has been submitted to a liquid composition. In one embodiment of the invention, the liquid composition can be DMSO (dimethylsulfoxide) or another organic solvent. In one embodiment, the liquid composition can be water or an aqueous solution or an aqueous suspension or a buffered aqueous solution. The liquid composition may comprise water and at least one surfactant and/or at least one builder. In one embodiment, the liquid composition can be wash liquor comprising at least one surfactant. In one embodiment, the liquid composition can comprise water and a detergent composition such as a laundry detergent composition, a dishwashing composition or a detergent composition for hard surface cleaning.

In one embodiment, the first surface is submitted to a liquid composition such as a wash liquor optionally comprising at least one enzyme. It is thereby possible, by detecting the presence of fluorophore, to detect how much composition that is left on the first surface. When the wash liquor comprises at least one enzyme, it is possible, by detecting the presence of fluorophore, to detect how much composition that is left on the first surface after washing with a liquid composition comprising an enzyme. One further advantage is that the assay allows detecting the presence of fluorophore after washing with a wash liquor without enzymes and comparing with the presence of fluorophore after washing with a wash liquor with enzymes.

In one embodiment, the first surface and the second surface are submitted to a liquid composition simultaneously. In one embodiment, the liquid composition is a wash liquor optionally comprising at least one enzyme. One advantage by simultaneously washing the first and the second surface is that it is possible to detect the presence of fluorophore on the second surface after the washing step. This makes it possible to see if the composition applied on the first surface is re-deposited on the second surface during wash.

In one embodiment, the liquid composition does not comprise an optical brightener having an emission wavelength overlapping the emission wavelength of the fluorophore comprised in the composition.

Fluorophore molecules could be either utilized alone, or serve as a fluorescent motif of a functional system. Based on molecular complexity and synthetic methods, fluorophores could be generally classified into four categories: proteins and peptides, small organic compounds, synthetic oligomers and polymers, and multi-component systems.

In the present invention, the fluorophore is a small organic compound belonging to the following chemical families:
Xanthene derivatives including fluorescein, rhodamine, Oregon green, eosin, and Texas red
Cyanine derivatives including cyanine, indocarbocyanine, oxacarbocyanine, thiacarbocyanine, and merocyanine
Naphthalene derivatives including dansyl and prodan derivatives)
Coumarin derivatives
Oxadiazole derivatives including pyridyloxazole, nitrobenzoxadiazole and benzoxadiazole
Anthracene derivatives including anthraquinones, including DRAQ5, DRAQ7 and CyTRAK Orange
Pyrene derivatives including cascade blue etc.
Oxazine derivatives including Nile red, Nile blue, cresyl violet, oxazine 170 etc.
Acridine derivatives including proflavin, acridine orange, acridine yellow etc.
Arylmethine derivatives including auramine, crystal violet, malachite green
Tetrapyrrole derivatives including porphin, phthalocyanine, bilirubin and
anthranilic acid derivatives including N-methylanthraniloyl derivatives formed by reaction of nucleophiles such as alcohols and amines with MIA (N-methylisatoic anhydride)

The fluorophore is comprised in the composition. In one embodiment, the fluorophore is labelled (covalently or non-covalently bound (or attached)) to the composition or labelled to at least one component of the composition. The fluorophore can be comprised in the composition by being covalently attached to at least one component of the composition or via a linker. Typically the fluorophore or the linker are connected to functional group(s) such as alcohol (—OH), amino (—NH2 or —NHR), thiol (—SH), carboxylate (—COOH) and aldehyde (—CHO) group(s) present in the at least one component of the composition. Typically, the fluorescence labeling step is conducted by reacting the fluorophore, the fluorophore containing an activated group or the fluorophore containing a linker with an activated group to functional groups in the component to be labeled. Activated groups of fluorophores or linkers can be selected from the group consisting of triazines or chlorotriazines (such as dichlorotriazines), sulfatoethyl dyes, active esters (including anhydrides such as N-methylisatoic anhydride (MIA) and succinimidyl esters) hydrazide, maleimide and arylboronic acids.

In one embodiment of the invention, the composition comprises a fluorophore selected from the group of antranilic acid derivatives. In one embodiment the fluorophore is a N-methylanthraniloyl ester formed by reaction of an alcohol with N-methylisatoic anhydride (MIA).

| | Absorbance | Emission | |
|---|---|---|---|
| Fluorophore | wavelength | wavelength | Visible color |
| N-methylanthraniloyl | 350-360 | 430-450 | Blue |
| ester (derived from | | | |
| MIA) | | | |
| N-methylanthraniloyl | 350-360 | 430-450 | Blue |
| amide (derived from | | | |
| MIA) | | | |
| hydroxycoumarin | 325 | 386 | Blue |
| methoxycoumarin | 360 | 410 | Blue |
| Alexa fluor | 345 | 442 | Blue |
| aminocoumarin | 350 | 445 | Blue |
| Cy 2 | 490 | 510 | Green (dark) |
| FAM | 495 | 516 | Green (dark) |
| Alexa fluor 488 | 494 | 517 | Green (light) |
| Fluorescein FITC | 495 | 518 | Green (light) |
| DTAF (5-(4,6- | 495 | 519 | Green (light) |
| dichlorotriazinyl) | | | |
| aminofluorescein) | | | |
| NBD (4-Chloro-7- | 420 | 540 | Green (light) |
| nitrobenzofurazan) | | | |
| Alexa fluor 430 | 430 | 545 | Green (light) |
| Alexa fluor 532 | 530 | 555 | Green (light) |
| HEX | 535 | 556 | Green (light) |
| Cy3 | 550 | 570 | Yellow |
| TRITC | 547 | 572 | Yellow |
| Alexa fluor 546 | 556 | 573 | Yellow |
| Alexa fluor 555 | 556 | 573 | Yellow |
| R-phycoerythrin (PE) | 480; 565 | 578 | Yellow |
| Rhodamine Red-X | 560 | 580 | Orange |
| Tamara | 565 | 580 | Red |
| Cy3.5 581 | 581 | 596 | Yellow |
| Rox | 575 | 602 | Yellow |
| Alexa fluor 568 | 578 | 603 | Yellow |
| Red 613 | 480; 565 | 613 | Yellow |
| Texas Red | 615 | 615 | Yellow |
| Alexa fluor 594 | 590 | 617 | Yellow |
| Alexa fluor 633 | 621 | 639 | Yellow |
| Allophycocyanin | 650 | 660 | Yellow |
| Alexa fluor 633 | 650 | 668 | Yellow |
| Cy5 | 650 | 670 | Yellow |
| Alexa fluor 660 | 663 | 690 | Yellow |
| Cy5.5 | 675 | 694 | Yellow |
| TruRed | 490; 675 | 695 | Yellow |
| Alexa fluor 680 | 679 | 702 | Yellow |
| Cy7 | 743 | 770 | Yellow |
| DAPI | 345 | 455 | Blue |
| Hoechst 33258 | 345 | 478 | Blue |
| SYTOX blue | 431 | 480 | Blue |
| Hoechst 33342 | 343 | 483 | Blue |
| YOYO-1 | 509 | 509 | Green |
| SYTOX green | 504 | 533 | Green |
| TOTO 1, TO-PRO-1 | 509 | 533 | Green |
| SYBR-safe | 502 | 530 | Green |
| SYTOX orange | 547 | 570 | Yellow |
| Chromomycin A3 | 445 | 575 | Yellow |
| Mithamycin | 445 | 575 | Yellow |
| propidium iodide | 536 | 617 | Red |
| ethidium bromide | 493 | 620 | Red |

The fluorophore used may have an absorbance wavelength in the range of 200-810 nm and an emission wavelength in the range of 200-810 nm. In one embodiment the fluorophore has an absorbance wavelength in the range of 224-804 nm and an emission wavelength in the range of 224-804 nm.

In one embodiment of the invention, the fluorophore has an absorbance wavelength in the range of 320-400 nm, such as in the range of 325-360nm, in the range of 340-360 nm or in the range of 350-360 nm.

In one embodiment of the invention, the fluorophore has absorbance wavelength in the range of 400-540 nm, such as in the range of 490-535, in the range of 490-494 nm or in the range of 495-535 nm.

In one embodiment of the invention, the fluorophore has an absorbance wavelength in the range of 550-560, such as in the range of 350-550 nm, in the range of 490-535 nm or in the range of 565-743 nm.

In one embodiment of the invention, the fluorophore has an absorbance wavelength in the range of 565-810, such as in the range of 565-743 nm.

In one embodiment of the invention, the fluorophore can be selected from the group consisting of N-methylanthraniloyl ester (derived from MIA), N-methylanthraniloyl amide (derived from MIA) anthraniloyl derivatives, N-methyl isatoic anhydride (MIA), hydroxycoumarin, methoxycoumarin, Alexa fluor, aminocoumarin, Cy 2, FAM, Alexa fluor 488, Fluorescein FITC, DTAF, NBD, Alexa fluor 430, Alexa fluor 532, HEX, Cy3, TRITC, Alexa fluor 546, Alexa fluor 555, R-phycoerythrin (PE), Rhodamine Red-X, Tamara, Cy3.5 581, Rox, Alexa fluor 568, Red 613, Texas Red, Alexa fluor 594, Alexa fluor 633, Allophycocyanin, CyS, Alexa fluor 660, Cy5.5, TruRed, Alexa fluor 680, Cy7, DAPI and Hoechst 33258, SYTOX blue, Hoechst 33342, YOYO-1, SYTOX green, TOTO 1 TO-PRO-1, SYTOX orange, Chromomycin A3, Mithamycin, propidium iodide and ethidium bromide. In a preferred embodiment the fluorophore is a N-methylanthraniloyl ester (derived from MIA), or DAPI.

In one embodiment of the invention, the composition comprising the fluorophore further comprises organic and/or inorganic material. The composition can comprise a raw material, a food product, material secreted from human or animal body, particulate material or a mixture hereof.

In one embodiment, the composition comprises a food product selected from the group consisting of polysaccharides, proteins and lipids. The food product can be selected from the group consisting of mashed potatoes, oatmeal porridge, dairy products and meat products.

In one embodiment, the composition can comprise a polysaccharide selected from the group consisting of starch, glycogen, arabinoxylan, cellulose, chitin, pectin, xanthan, carrageenan, arabinogalactan, xyloglucan, xylan, glucuronoxylan, galactan, glucan, arabinan, mannan, glucomannan, galactomannan, xyloglucan, arabinogalactan and pullulan or a mixture thereof. The polysaccharide can be in a food product or in other products, e.g. drug products.

In one embodiment, the composition can comprise a material secreted from human or animal body such as blood, sweat, lipid, grease, sebum, drool, vomit, microorganisms, odor, DNA or mixtures hereof.

Some material secreted from human or animal body may be present in a biofilm attached to a surface. A biofilm is any group of microorganisms in which cells stick to each other on a surface, such as a textile, dishware or hard surface. These adherent cells are frequently embedded within a self-produced matrix of extracellular polymeric substance (EPS). Biofilm EPS is a polymeric conglomeration generally composed of extracellular DNA, proteins, polysaccharides and microorganism. Biofilms may form on living or non-living surfaces. The microbial cells growing in a biofilm are physiologically distinct from planktonic cells of the same organism, which, by contrast, are single-cells that may float or swim in a liquid medium.

Bacteria living in a biofilm usually have significantly different properties from free-floating bacteria of the same species, as the dense and protected environment of the film allows them to cooperate and interact in various ways. One benefit of this environment is increased resistance to detergents and antibiotics, as the dense extracellular matrix and the outer layer of cells protect the interior of the community.

The liquid composition can comprise water. In one embodiment the liquid composition is a buffered aqueous solution. In one embodiment the liquid composition further comprises a surfactant. The liquid composition can be a wash liquor comprising a detergent composition, a dishwashing composition or a composition for cleaning hard surfaces.

In one embodiment, the liquid composition is DMSO or another organic solvent.

The composition can be a soil or a stain. Soil or stains present on items such as textile, dishware or hard surfaces are very often caused by spilling of food stuff. One application of the assay is for testing the presence of a fluorophore after the washing of a textile, a dishware or a hard surface. A composition comprising the fluorophore can be applied to such surface. This reflects the soiling of a surface. The surface is then submitted to a liquid composition, which can be water or a liquid composition comprising a surfactant or a detergent composition. This reflects the washing of the textile, the dishware or the hard surface. The surface is then exposed to light having a wavelength within +/−30 nm of the absorbance wavelength of the fluorophore whereby the fluorophore emits light that can be detected by human eye or detected according to assay I.

Thereby, it is possible to test how much soil that is removed during the washing with enzymes of a textile, a dishware or a hard surface. Further, it is possible to test how much soil that remains on the surface e.g. by using the quantification method described in Assay I. In one embodiment, it is possible to compare the presence of fluorophore before and after wash and thereby determine how much soil that has been removed.

The surface is submitted to a liquid composition, which can be water or a liquid composition comprising a surfactant or a detergent composition.

The detergent composition may comprise one or more surfactants, which may be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 1% to about 40%, or about 3% to about 20%, or about 3% to about 10%. The surfactant(s) is chosen based on the desired cleaning application, and may include any conventional surfactant(s) known in the art.

When included therein, the detergent will usually contain from about 1% to about 40% by weight of an anionic surfactant, such as from about 5% to about 30%, including from about 5% to about 15%, or from about 15% to about 20%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), and combinations thereof.

When included therein, the detergent will usually contain from about from about 1% to about 40% by weigh of a cationic surfactant, for example from about 0.5% to about 30%, in particular from about 1% to about 20%, from about 3% to about 10%, such as from about 3% to about 5%, from about 8% to about 12% or from about 10% to about 12%. Non-limiting examples of cationic surfactants include alkyldimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, ester quats, and combinations thereof.

When included therein, the detergent will usually contain from about 0.2% to about 40% by weight of a nonionic surfactant, for example from about 0.5% to about 30%, in particular from about 1% to about 20%, from about 3% to about 10%, such as from about 3% to about 5%, from about 8% to about 12%, or from about 10% to about 12%. Non-limiting examples of nonionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof.

When included therein, the detergent will usually contain from about 0% to about 40% by weight of a semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, and combinations thereof.

When included therein, the detergent will usually contain from about 0% to about 40% by weight of a zwitterionic surfactant. Non-limiting examples of zwitterionic surfactants include betaines such as alkyldimethylbetaines, sulfobetaines, and combinations thereof.

The detergent composition can further comprise a hydrotrope. A hydrotrope is a compound that solubilises hydrophobic compounds in aqueous solutions (or oppositely, polar substances in a non-polar environment). Typically, hydrotropes have both hydrophilic and a hydrophobic character (so-called amphiphilic properties as known from surfactants); however the molecular structure of hydrotropes generally do not favor spontaneous self-aggregation, see e.g. review by Hodgdon and Kaler (2007), Current Opinion in Colloid & Interface Science 12: 121-128. Hydrotropes do not display a critical concentration above which self-aggregation occurs as found for surfactants and lipids forming miceller, lamellar or other well defined meso-phases. Instead, many hydrotropes show a continuous-type aggregation process where the sizes of aggregates grow as concentration increases. However, many hydrotropes alter the phase behavior, stability, and colloidal properties of systems containing substances of polar and non-polar character, including mixtures of water, oil, surfactants, and polymers. Hydrotropes are classically used across industries from pharma, personal care, food, to technical applications. Use of hydrotropes in detergent compositions allow for example more concentrated formulations of surfactants (as in the process of compacting liquid detergents by removing water) without inducing undesired phenomena such as phase separation or high viscosity.

The detergent may contain 0-10% by weight, for example 0-5% by weight, such as about 0.5 to about 5%, or about 3% to about 5%, of a hydrotrope. Any hydrotrope known in the art for use in detergents may be utilized. Non-limiting examples of hydrotropes include sodium benzenesulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

The detergent composition may contain about 0-65% by weight, such as about 5% to about 50% of a detergent builder or co-builder, or a mixture thereof. In a dish wash detergent, the level of builder is typically 40-65%, particularly 50-65%. The builder and/or co-builder may particularly be a chelating agent that forms water-soluble complexes with Ca and Mg. Any builder and/or co-builder known in the art for use in laundry/ADW/hard surface cleaning detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as 2,2′-iminodiethan-1-ol), triethanolamine (TEA, also known as 2,2′,2″-nitrilotriethan-1-ol), and (carboxymethyl)inulin (CMI), and combinations thereof.

The detergent composition may also contain 0-50% by weight, such as about 5% to about 30%, of a detergent co-builder. The detergent composition may include include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra(methylenephosphonic acid) (EDTMPA), diethylenetriaminepentakis(methylenephosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA), N-(2-hydroxyethypethylenediamine-N,N′,N″-triacetic acid (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 09/102854, U.S. Pat. No. 5,977,053

The detergent composition may contain 0-30% by weight, such as about 1% to about 20%, of a bleaching system. Any bleaching system known in the art for use in laundry/ADW/hard surface cleaning detergents may be utilized. Suitable bleaching system components include bleaching catalysts, photobleaches, bleach activators, sources of hydrogen peroxide such as sodium percarbonate, sodium perborates and hydrogen peroxide-urea (1:1), preformed peracids and mixtures thereof. Suitable preformed peracids include, but are not limited to, peroxycarboxylic acids and salts, diperoxydicarboxylic acids, perimidic acids and salts, peroxymonosulfuric acids and salts, for example, Oxone (R), and mixtures thereof. Non-limiting examples of bleaching systems include peroxide-based bleaching systems, which may comprise, for example, an inorganic salt, including alkali metal salts such as sodium salts of perborate (usually mono- or tetra-hydrate), percarbonate, persulfate, perphosphate, persilicate salts, in combination with a peracid-forming bleach activator.

The term bleach activator is meant herein as a compound which reacts with hydrogen peroxide to form a peracid via perhydrolysis. The peracid thus formed constitutes the activated bleach. Suitable bleach activators to be used herein include those belonging to the class of esters, amides, imides or anhydrides. Suitable examples are tetraacetylethylenediamine (TAED), sodium 4-[(3,5,5-trimethylhexanoyl)oxy]benzene-1-sulfonate (ISONOBS), 4-(dodecanoyloxy)benzene-1-sulfonate (LOBS), 4-(decanoyloxy)benzene-1-sulfonate, 4-(decanoyloxy)benzoate (DOBS or DOBA), 4-(nonanoyloxy)benzene-1-sulfonate (NOBS), and/or those disclosed in WO98/17767. A particular family of bleach activators of interest was disclosed in EP624154 and particularly preferred in that family is acetyl triethyl citrate (ATC). ATC or a short chain triglyceride like triacetin has the advantage that it is environmentally friendly Furthermore acetyl triethyl citrate and triacetin have good hydrolytical stability in the product upon storage and are efficient bleach activators. Finally ATC is multifunctional, as the citrate released in the perhydrolysis reaction may function as a builder. Alternatively, the bleaching system may comprise peroxyacids of, for example, the amide, imide, or sulfone type. The bleaching system may also comprise peracids such as 6-(phthalimido)peroxyhexanoic acid (PAP). The bleaching system may also include a bleach catalyst. In some embodiments the bleach component may be an organic catalyst selected from the group consisting of organic catalysts having the following formulae:

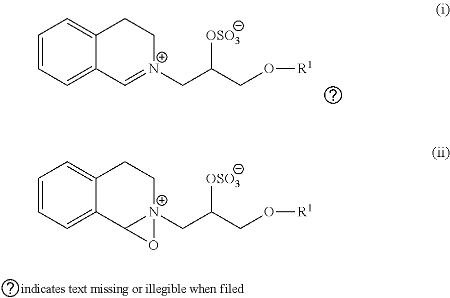

(iii) and mixtures thereof;

wherein each R¹ is independently a branched alkyl group containing from 9 to 24 carbons or linear alkyl group containing from 11 to 24 carbons, preferably each R¹ is independently a branched alkyl group containing from 9 to 18 carbons or linear alkyl group containing from 11 to 18 carbons, more preferably each R¹ is independently selected from the group consisting of 2-propylheptyl, 2-butyloctyl, 2-pentylnonyl, 2-hexyldecyl, dodecyl, tetradecyl, hexadecyl, octadecyl, isononyl, isodecyl, isotridecyl and isopentadecyl. Other exemplary bleaching systems are described, e.g. in WO2007/087258, WO2007/087244, WO2007/087259, EP1867708 (Vitamin K) and WO2007/087242. Suitable photobleaches may for example be sulfonated zinc or aluminium phthalocyanines.

Preferably the bleach component comprises a source of peracid in addition to bleach catalyst, particularly organic bleach catalyst. The source of peracid may be selected from (a) pre-formed peracid; (b) percarbonate, perborate or persulfate salt (hydrogen peroxide source) preferably in combination with a bleach activator; and (c) perhydrolase enzyme and an ester for forming peracid in situ in the presence of water in a textile or hard surface treatment step.

The detergent composition may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated.

The detergent compositions may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO2005/03274, WO2005/03275, WO2005/03276 and EP1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g. WO 2007/087257 and WO2007/087243.

The detergent compositions can also contain dispersants. In particular, powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

The detergent compositions may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

The detergent compositions may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore random graft co-polymers are suitable soil release polymers. Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose derivatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

The detergent compositions may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

The detergent compositions may also include one or more rheology modifiers, structurants or thickeners, as distinct from viscosity reducing agents. The rheology modifiers are selected from the group consisting of non-polymeric crystalline, hydroxy-functional materials, polymeric rheology modifiers which impart shear thinning characteristics to the aqueous liquid matrix of a liquid detergent composition. The rheology and viscosity of the detergent can be modified and adjusted by methods known in the art, for example as shown in EP 2169040.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

In one embodiment of the invention, the liquid composition comprises at least one enzyme. The enzyme can be selected from the group consisting of hemicellulases, peroxidases, proteases, cellulases, xylanases, lipases, phospholipases, esterases, cutinases, pectinases, mannanases, pectate lyases, keratinases, reductases, oxidases, phenoloxidases, lipoxygenases, ligninases, pullulanases, tannases, pentosanases, malanases, β-glucanases, arabinosidases, hyaluronidase, chondroitinase, laccase, DNase, chlorophyllases, amylases, perhydrolases, peroxidases, xanthanase and mixtures thereof. In one embodiment the enzyme is an amylase.

Suitable mannanases include those of bacterial or fungal origin. Chemically or genetically modified mutants are included. The mannanase may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 1999/064619. A commercially available mannanase is Mannaway (Novozymes A/S).

Suitable cellulases include complete cellulases or mono-component endoglucanases of bacterial or fungal origin. Chemically or genetically modified mutants are included. The cellulase may for example be a mono-component or a mixture of mono-component endo-1,4-beta-glucanase often just termed endoglucanases. Suitable cellulases include a fungal cellulase from Humicola insolens (U.S. Pat. No. 4,435,307) or from Trichoderma, e.g. T. reesei or T. viride. Examples of cellulases are described in EP 0 495 257. Other suitable cellulases are from Thielavia e.g. Thielavia terrestris as described in WO 96/29397 or Fusarium oxysporum as described in WO 91/17244 or from Bacillus as described in, WO 02/099091 and JP 2000210081. Other examples are cellulase variants such as those described in WO 94/07998, EP 0 531 315, U.S. Pat. No. 5,457,046, U.S. Pat. No. 5,686,593, U.S. Pat. No. 5,763,254, WO 95/24471, WO 98/12307 Commercially available cellulases include Carezyme®, Celluzyme®, Celluclean®, Celluclast® and Endolase®; Renozyme®; Whitezyme® (Novozymes A/S) Puradax®, Puradax HA, and Puradax EG (available from Genencor).

Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257. Commercially available peroxidases include Guardzyme™ (Novozymes A/S).

Suitable proteases include those of bacterial, fungal, plant, viral or animal origin e.g. vegetable or microbial origin. Microbial origin is preferred. Chemically modified or protein engineered mutants are included. It may be an alkaline protease, such as a serine protease or a metalloprotease. A serine protease may for example be of the S1 family, such as trypsin, or the S8 family such as subtilisin. A metalloproteases protease may for example be a thermolysin from e.g. family M4 or other metalloprotease such as those from M5, M7 or M8 families.

The term “subtilases” refers to a sub-group of serine protease according to Siezen et al., Protein Engng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 sub-divisions, i.e. the Subtilisin family, the Thermitase family, the Proteinase K family, the Lantibiotic peptidase family, the Kexin family and the Pyrolysin family.

Examples of subtilases are those derived from Bacillus such as Bacillus lentus, B. alkalophilus, B. subtilis, B. amyloliquefaciens, Bacillus pumilus and Bacillus gibsonii described in; U.S. Pat. No. 7,262,042 and WO 09/021867, and subtilisin lentus, subtilisin Novo, subtilisin Carlsberg, Bacillus licheniformis, subtilisin BPN', subtilisin 309, subtilisin 147 and subtilisin 168 described in WO 89/06279 and protease PD138 described in (WO 93/18140). Other useful proteases may be those described in WO 92/175177, WO 01/016285, WO 02/026024 and WO 02/016547. Examples of trypsin-like proteases are trypsin (e.g. of porcine or bovine origin) and the Fusarium protease described in WO 89/06270, WO 94/25583 and WO 05/040372, and the chymotrypsin proteases derived from Cellumonas described in WO 05/052161 and WO 05/052146.

A further preferred protease is the alkaline protease from Bacillus lentus DSM 5483, as described for example in WO 95/23221, and variants thereof which are described in WO 92/21760, WO 95/23221, EP 1921147 and EP 1921148.

Examples of metalloproteases are the neutral metalloprotease as described in WO 07/044993 (Genencor Int.) such as those derived from Bacillus amyloliquefaciens.

Examples of useful proteases are the variants described in: WO 92/19729, WO 96/034946, WO 98/20115, WO 98/20116, WO 99/011768, WO 01/44452, WO 03/006602, WO 04/03186, WO 04/041979, WO 07/006305, WO 11/036263, WO 11/036264, especially the variants with substitutions in one or more of the following positions: 3, 4, 9, 15, 27, 36, 57, 68, 76, 87, 95, 96, 97, 98, 99, 100, 101, 102, 103, 104, 106, 118, 120, 123, 128, 129, 130, 160, 167, 170, 194, 195, 199, 205, 206, 217, 218, 222, 224, 232, 235, 236, 245, 248, 252 and 274 using the BPN' numbering. More preferred the subtilase variants may comprise the mutations: S3T, V4I, S9R, A15T, K27R, *36D, V68A, N76D, N87S,R, *97E, A98S, S99G,D,A, S99AD, S101G,M,R S103A, V104I,Y,N, S106A, G118V,R, H120D,N, N123S, S128L, P129Q, S130A, G160D, Y167A, R170S, A194P, G195E, V199M, V205I, L217D, N218D, M222S, A232V, K235L, Q236H, Q245R, N252K, T274A (using BPN' numbering).

Suitable commercially available protease enzymes include those sold under the trade names Alcalase®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Neutrase®, Everlase® and Esperase® (Novozymes A/S), those sold under the tradename Maxatase®, Maxacal®, Maxapem®, Purafect®, Purafect Prime®, Purafect MA®, Purafect Ox®, Purafect OxP®, Puramax®, Properase®, FN2®, FN3®, FN4®, Excellase®, Eraser®, Opticlean® and Optimase® (Danisco/DuPont), Axapem™ (Gist-Brocases N.V.), BLAP (sequence shown in FIG. 29 of U.S. Pat. No. 5,352,604) and variants hereof (Henkel AG) and KAP (Bacillus alkalophilus subtilisin) from Kao.

Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP 258068 and EP 305216, cutinase from Humicola, e.g. H. insolens (WO 96/13580), lipase from strains of Pseudomonas (some of these now renamed to Burkholderia), e.g. P. alcaligenes or P. pseudoalcaligenes (EP 218272), P. cepacia (EP 331376), P. sp. strain SD705 (WO 95/06720 & WO 96/27002), P. wisconsinensis (WO 96/12012), GDSL-type Streptomyces lipases (WO 10/065455), cutinase from Magnaporthe grisea (WO 10/107560), cutinase from Pseudomonas mendocina (U.S. Pat. No. 5,389,536), lipase from Thermobifida fusca (WO 11/084412), Geobacillus stearothermophilus lipase (WO 11/084417), lipase from Bacillus subtilis (WO 11/084599), and lipase from Streptomyces griseus (WO 11/150157) and S. pristinaespiralis (WO 12/137147).

Other examples are lipase variants such as those described in EP 407225, WO 92/05249, WO 94/01541, WO 94/25578, WO 95/14783, WO 95/30744, WO 95/35381, WO 95/22615, WO 96/00292, WO 97/04079, WO 97/07202, WO 00/34450, WO 00/60063, WO 01/92502, WO 07/87508 and WO 09/109500.

Preferred commercial lipase products include include Lipolase™, Lipex™; Lipolex™ and Lipoclean™ (Novozymes A/S), Lumafast (originally from Genencor) and Lipomax (originally from Gist-Brocades).

Still other examples are lipases sometimes referred to as acyltransferases or perhydrolases, e.g. acyltransferases with homology to Candida antarctica lipase A (WO 10/111143), acyltransferase from Mycobacterium smegmatis (WO 05/56782), perhydrolases from the CE 7 family (WO 09/67279), and variants of the M. smegmatis perhydrolase in particular the S54V variant used in the commercial product Gentle Power Bleach from Huntsman Textile Effects Pte Ltd (WO 10/100028).

Suitable amylases which can be used together with the enzyme/variant/blend of enzymes of the invention may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, I201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions: M197T; H156Y+A181T+N190F+A209V+Q264S; or G48A+T49I+G107A+H156Y+A181T+N190F+I201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, I206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/023873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions: N128C+K178L+T182G+Y305R+G475K; N128C+K178L+T182G+F202Y+Y305R+D319T+G475K; S125A+N128C+K178L+T182G+Y305R+G475K; or S125A+N128C+T131I+T165I+K178L+T182G+Y305R+G475K wherein the variants are C-terminally truncated and optionally further comprises a substitution at position 243 and/or a deletion at position 180 and/or position 181.

Further suitable amylases are amylases having SEQ ID NO: 1 of WO13184577 or variants having 90% sequence identity to SEQ ID NO: 1 thereof. Preferred variants of SEQ ID NO: 1 are those having a substitution, a deletion or an insertion in one of more of the following positions: K176, R178, G179, T180, G181, E187, N192, M199, 1203, S241, R458, T459, D460, G476 and G477. More preferred variants of SEQ ID NO: 1 are those having the substitution in one of more of the following positions: K176L, E187P, N192FYH, M199L, I203YF, S241QADN, R458N, T459S, D460T, G476K and G477K and/or deletion in position R178 and/or S179 or of T180 and/or G181. Most preferred amylase variants of SEQ ID NO: 1 are those having the substitutions: E187P+I203Y+G476K; E187P+I203Y+R458N+T459S+D460T+G476K, wherein the variants optionally further comprises a substitution at position 241 and/or a deletion at position 178 and/or position 179.

Further suitable amylases are amylases having SEQ ID NO: 1 of WO 10104675 or variants having 90% sequence identity to SEQ ID NO: 1 thereof. Preferred variants of SEQ ID NO: 1 are those having a substitution, a deletion or an insertion in one of more of the following positions: N21, D97, V128 K177, R179, S180, I181, G182, M200, L204, E242, G477 and G478. More preferred variants of SEQ ID NO: 1 are those having the substitution in one of more of the following positions: N21D, D97N, V128I K177L, M200L, L204YF, E242QA, G477K and G478K and/or deletion in position R179 and/or S180 or of 1181 and/or G182. Most preferred amylase variants of SEQ ID NO: 1 are those having the substitutions: N21D+D97N+V128I, wherein the variants optionally further comprises a substitution at position 200 and/or a deletion at position 180 and/or position 181.

Other suitable amylases are the alpha-amylase having SEQ ID NO: 12 in WO 01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO 2011/098531, WO 2013/001078 and WO 2013/001087.

Commercially available amylases are Duramyl™, Termamyl™, Fungamyl™, Stainzyme™, Stainzyme Plus™, Natalase™, Liquozyme X and BAN™ (from Novozymes A/S), and Rapidase™ Purastar™/Effectenz™, Powerase, Preferenz S1000, Preferenz S100 and Preferenz S110 (from Genencor International Inc./DuPont).

Suitable peroxidases (EC 1.11.1.7) include those of plant, bacterial or fungal origin. Chemically modified or protein engineered mutants are included. Examples of useful peroxidases include peroxidases from Coprinopsis, e.g., from C. cinerea (EP 179,486), and variants thereof as those described in WO 93/24618, WO 95/10602, and WO 98/15257.

Peroxidases also include a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions.

In an embodiment, the haloperoxidase of the invention is a chloroperoxidase. Preferably, the haloperoxidase is a vanadium haloperoxidase, i.e., a vanadate-containing haloperoxidase. In a preferred method of the present invention the vanadate-containing haloperoxidase is combined with a source of chloride ion.

Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis. Haloperoxidases have also been isolated from bacteria such as Pseudomonas, e.g., P. pyrrocinia and Streptomyces, e.g., S. aureofaciens.

In a preferred embodiment, the haloperoxidase is derivable from Curvularia sp., in particular Curvularia verruculosa or Curvularia inaequalis, such as C. inaequalis CBS 102.42 as described in WO 95/27046; or C. verruculosa CBS 147.63 or C. verruculosa CBS 444.70 as described in WO 97/04102; or from Drechslera hartlebii as described in WO 01/79459, Dendryphiella salina as described in WO 01/79458, Phaeotrichoconis crotalarie as described in WO 01/79461, or Geniculosporium sp. as described in WO 01/79460.

An oxidase according to the invention include, in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5).

Preferred laccase enzymes are enzymes of microbial origin. The enzymes may be derived from plants, bacteria or fungi (including filamentous fungi and yeasts).

Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radiata (WO 92/01046), or Coriolus, e.g., C. hirsutus (JP 2238885).

Suitable examples from bacteria include a laccase derivable from a strain of Bacillus.

A laccase derived from Coprinopsis or Myceliophthora is preferred; in particular a laccase derived from Coprinopsis cinerea, as disclosed in WO 97/08325; or from Myceliophthora thermophila, as disclosed in WO 95/33836.

The detergent enzyme(s) may be included in a detergent composition by adding separate additives containing one or more enzymes, or by adding a combined additive comprising all of these enzymes. A detergent additive of the invention, i.e., a separate additive or a combined additive, can be formulated, for example, as a granulate, liquid, slurry, etc. Preferred detergent additive formulations are granulates, in particular non-dusting granulates, liquids, in particular stabilized liquids, or slurries.

Non-dusting granulates may be produced, e.g. as disclosed in U.S. Pat. Nos. 4,106,991 and 4,661,452 and may optionally be coated by methods known in the art. Examples of waxy coating materials are polyethyleneglycol (PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216.

When an enzyme is used in the liquid composition, it is possible to test how much fluorophore that is present on the surface after the surface has been submitted to the liquid composition comprising the enzyme. As described above, the surface can be exposed to light having a wavelength within +/−30 nm of the absorbance wavelength of the fluorophore so the fluorophore emits light that can be detected by human eye or detected according to assay I or Assay IV. Thereby it is possible to test how much soil that is removed during the washing with enzymes of a textile, a dishware or a hard surface. Further it is possible to test how much soil that remains on the surface. In one embodiment it is possible to compare the presence of fluorophore before and after wash and thereby determine how much soil that has been removed.

In one embodiment of the invention, the composition is applied to the first surface, which is then together with a second surface submitted to a liquid composition. The composition can be a soil or a stain. One application of this assay is for testing the redeposition of soil during a washing step by testing the presence of a fluorophore after the washing of a textile, a dishware or a hard surface. Redeposition of the composition from the first surface to the second surface can be detected by exposing exposed the second surface to light having a wavelength within +/−30 nm of the absorbance wavelength of the fluorophore so the fluorophore emits light that can be detected by human eye or detected according to assay I. Thereby it is possible to test how much soil that is deposited during the washing of a textile, a dishware or a hard surface. The assay can be carried out as describe above and optionally with at least one enzyme present in the liquid composition.

If the purpose of the assay and method is to test how much fluorophore that is present after the surface has been submitted to a liquid composition comprising at least one enzyme, the composition should comprise an amount of fluorophore that allows the enzyme to degrade or partially degrade the composition to which the fluorophore is attached (covalently or non-covalently).

In one embodiment of the invention, the composition comprises a polysaccharide comprising at least one fluorophore per 300 monosaccharides, such as at least one fluorophore per 250 monosaccharides, at least one fluorophore per 200 monosaccharides, at least one fluorophore per 150 monosaccharides, at least one fluorophore per 100 monosaccharides,at least one fluorophore per 50 monosaccharides or at least one fluorophore per 25 monosaccharides.

In one embodiment of the invention, the composition comprises a protein comprising at least one fluorophore per 300 amino acids, such as at least one fluorophore per 250 amino acids, at least one fluorophore per 200 amino acids, at least one fluorophore per 150 amino acids, at least one fluorophore per 100 amino acids or at least one fluorophore per 50 amino acids or at least one fluorophore per 25 amino acids.

In one embodiment of the invention, the composition comprises a lipid comprising cholesterol, free fatty acids, diglycerides, triglycerides and/or phospholipids.

In one embodiment of the invention, the surface can be rinsed with water after being submitted to the liquid composition, where the water optionally comprises a fabric conditioner.

The first and/or the second surface may be exposed to light having a wavelength within +/−29 nm of the absorbance wavelength of the fluorophore, such as within +/−28 nm, within +/−27 nm, within +/−26 nm, within +/−25 nm, within +/−24 nm, within +/−23 nm, within +/−22 nm, within +/−21 nm, within +/−20 nm, within +/−19 nm, within +/−18 nm, within +/−17 nm, within +/−16 nm, within +/−15 nm, within +/−14 nm, within +/−13 nm, within +/−12 nm, within +/−11 nm, within +/−10 nm, within +/−9 nm, within +/−8 nm, within +/−7 nm, within +/−6 nm, within +/−5 nm, within +/−4 nm, within +/−3 nm, within +/−2 nm or within +/−1 nm of the maximum absorbance wavelength of the fluorophore.

In one embodiment, the first and/or the second surface may be exposed to light having a wavelength corresponding to the absorbance wavelength of the fluorophore.

In one embodiment of the invention, the surface is exposed to light having a wavelength in the range of 200-900 nm. In one embodiment the first and/or the second surface is exposed to light having a wavelength in the range of 295-380 nm, such as in the range of 325-350 nm. In one embodiment the first and/or the second surface is exposed to light having a wavelength in the range of 380-565 nm, such as in the range of 490-535 nm. In one embodiment the first and/or the second surface is exposed to light having a wavelength in the range of 520-590 nm, such as in the range of 550-560 nm. In one embodiment the first and/or the second surface is exposed to light having a wavelength in the range of in the range of 535-773 nm, such as in the range of 565-743 nm.

The light emitted from fluorophore can be detected by human eye or detected by the method described in Assay I or Assay IV.

In one embodiment of the invention, the first surface is present on a textile, a dish ware or a hard surface. In one embodiment the second surface is present on a textile, a dish ware or a hard surface. In one embodiment the first and the second surface is textile e.g. two different clothes washed together or two different areas of same cloth.

In one embodiment, the light emitted from the fluorophore in the composition is invisible to human eye in day light.

In one embodiment, the assay and/or the method comprise the following steps:
a. Preparing a soil comprising a fluorophore having an absorbance wavelength and an emission wavelength,
b. applying the soil to the first surface,
c. submitting the first surface and/or the second surface to a wash liquor optionally comprising at least one enzyme,
d. optionally rinsing and/or drying the first surface and/or the second surface,
e. exposing the surface and/or the second surface to light having a wavelength within +/−30 nm of the absorbance wavelength of the fluorophore, and
f. detecting the light emitted from the fluorophore present on the first surface and/or the second surface.
   The invention is further summarized in the following paragraphs:
1. Assay for testing the presence of a fluorophore on a first and/or a second surface comprising the following steps:
   a. Preparing a composition comprising a fluorophore having an absorbance wavelength and an emission wavelength,
   b. applying the composition to the first surface,
   c. submitting the first surface and/or the second surface to a liquid composition,
   d. optionally rinsing and/or drying the first surface and/or the second surface,
   e. exposing the first surface and/or the second surface to light having a wavelength within +/−30 nm of the absorbance wavelength of the fluorophore, and
   f. detecting the light emitted from the fluorophore present on the first surface and/or the second surface.
2. Assay according to paragraph 1, wherein the fluorophore has an absorbance wavelength in the range of 200-810 nm and an emission wavelength in the range of 200-810 nm, such as the fluorophore has an absorbance wavelength in the range of 224-804 nm and an emission wavelength in the range of 224-804 nm.
3. Assay according to paragraphs 1 or 2, wherein the fluorophore has an absorbance wavelength in the range of 320-400 nm, such as in the range of 325-360nm, in the range of 340-360 nm or in the range of 350-360 nm.
4. Assay according to paragraphs 1 or 2, wherein the fluorophore has absorbance wavelength in the range of 400-540 nm, such as in the range of 490-535, in the range of 490-495 nm or in the range of 494-535 nm.
5. Assay according to paragraphs 1 or 2, wherein the fluorophore has an absorbance wavelength in the range of 550-560, such as in the range of 350-550 nm, in the range of 490-535 nm or in the range of 565-743 nm.
6. Assay according to paragraphs 1 or 2, wherein the fluorophore has an absorbance wavelength in the range of 565-810, such as in the range of 565-743 nm.
7. Assay according to any of the preceding paragraphs, wherein the fluorophore is selected from the group consisting of
   a) Xanthene derivatives including fluorescein, rhodamine, Oregon green, eosin and Texas red,
   b) Cyanine derivatives including cyanine, indocarbocyanine, oxacarbocyanine, thiacarbocyanine and merocyanine,
   c) Naphthalene derivatives including dansyl and prodan derivatives,
   d) Coumarin derivatives, including hydroxycoumarin, methoxycoumarin and aminocoumarin,
   e) Oxadiazole derivatives, including pyridyloxazole, nitrobenzoxadiazole and benzoxadiazole,
   f) Anthracene derivatives including anthraquinones, including DRAQ5, DRAQ7 and CyTRAK Orange,
   g) Pyrene derivatives including cascade blue,
   h) Oxazine derivatives including Nile red, Nile blue, cresyl violet, oxazine 170,
   i) Acridine derivatives including proflavin, acridine orange, acridine yellow,
   j) Arylmethine derivatives including auramine, crystal violet, malachite green
   k) Tetrapyrrole derivatives including porphin, phthalocyanine, bilirubin and
   l) Anthranilic acid derivatives including N-methylanthraniloyl derivatives formed by reaction of nucleophiles including alcohols and amines with MIA (N-methylisatoic anhydride).
8. Assay according to any of the preceding paragraphs, wherein the fluorophore is selected from the group consisting of anthranilic acid derivatives including N-methylanthraniloyl esters and amides formed by reaction of nucleophiles such as alcohols and amines with MIA (N-methylisatoic anhydride), N-methyl isatoic anhydride (MIA), hydroxycoumarin, methoxycoumarin, Alexa fluor, aminocoumarin, Cy 2, FAM, Alexa fluor 488, Fluorescein FITC, Alexa fluor 430, Alexa fluor 532, HEX, Cy3, TRITC, Alexa fluor 546, Alexa fluor 555, R-phycoerythrin (PE), Rhodamine Red-X, Tamara, Cy3.5 581, Rox, Alexa fluor 568, Red 613, Texas Red, Alexa fluor 594, Alexa fluor 633, Allophycocyanin, Cy5, Alexa fluor 660, Cy5.5, TruRed, Alexa fluor 680, Cy7, DAPI and Hoechst 33258, SYTOX blue, Hoechst 33342, YOYO-1, SYTOX green, TOTO 1 TO-PRO-1, SYTOX orange, Chromomycin A3, Mithamycin, propidium iodide and ethidium bromide.
9. Assay according to any of paragraphs 7 or 8, wherein the fluorophore is DAPI or N-methylanthraniloyl derivatives formed by reaction of nucleophiles such as alcohols and amines with MIA (N-methylisatoic anhydride).
10. Assay according to any of the preceding paragraphs, wherein the composition comprises organic and/or inorganic material.
11. Assay according to paragraph 10, wherein the composition comprises a raw material, a food product, material secreted from human or animal body, particulate material or a mixture hereof.
12. Assay according to any of paragraphs 10, wherein the composition comprises a food product comprising polysaccharides, proteins and/or lipids.
13. Assay according to any of paragraphs 10-12, wherein the composition is a food product selected from the group consisting of mashed potatoes, oatmeal porridge, dairy products and meat products.
14. Assay according to any of paragraphs 10-13, wherein the composition comprise a polysaccharide selected from the group consisting a polysaccharide selected from the group consisting of starch, glycogen, arabinoxylan, cellulose, chitin, pectin, xanthan, carrageenan, arabinogalactan, xyloglucan, xylan, glucuronoxylan, galactan, glucan, arabinan, mannan, glucomannan, galactomannan, xyloglucan, arabinogalactan and pullulan.
15. Assay according to any of paragraphs 10-14, wherein the composition comprise a material secreted from human or animal body such as blood, sweat, lipid, grease, sebum, drool, vomit, cells, microorganisms, odor, DNA and/or mixtures hereof.
16. Assay according to any of the preceding paragraphs, wherein the composition is a soil or a stain.
17. Assay according to any of the preceding paragraphs, wherein the fluorophore is labelled to at least one component in the composition comprising the fluorophore.
18. Assay according to any of the preceding paragraphs, wherein the liquid composition comprises water.
19. Assay according to any of the preceding paragraphs, wherein the liquid composition is a buffered aqueous solution.
20. Assay according to any of the preceding paragraphs, wherein the liquid composition further comprises a surfactant.
21. Assay according to any of the preceding paragraphs, wherein the liquid composition comprises a detergent composition, a dishwashing composition or a composition for cleaning hard surfaces.
22. Assay according to any of the preceding paragraphs, wherein the liquid composition is DMSO or another organic solvent.
23. Assay according to any of the preceding paragraphs, wherein the liquid composition further comprises at least one enzyme.
24. Assay according to paragraph 23, wherein the enzyme is selected from the group consisting of hemicellulases, peroxidases, proteases, cellulases, xylanases, lipases, phospholipases, esterases, cutinases, pectinases, mannanases, pectate lyases, keratinases, reductases, oxidases, phenoloxidases, lipoxygenases, ligninases, pullulanases, tannases, pentosanases, malanases, β-glucanases, arabinosidases, hyaluronidase, chondroitinase, laccase, DNase, chlorophyllases, amylases, perhydrolases, peroxidases, xanthanase and mixtures thereof.
25. Assay according to any of paragraphs 23-24, wherein the enzyme is an amylase.
26. Assay according to any of paragraphs 23-24, wherein the composition comprises an amount of fluorophore that allows the enzyme to degrade or partially degrade the composition.
27. Assay according to any of paragraphs 23-26, wherein composition comprises a polysaccharide comprising at least one fluorophore per 300 monosaccharides, such as at least one fluorophore per 250 monosaccharides, at least one fluorophore per 200 monosaccharides, at least one fluorophore per 150 monosaccharides, at least one fluorophore per 100 monosaccharides or at least one fluorophore per 50 monosaccharides.
28. Assay according to any of the preceding paragraphs, wherein the first surface and/or the second surface is rinsed with water after being submitted to the liquid composition, where the water optionally comprises a fabric conditioner.
29. Assay according any of the preceding paragraphs, wherein the first and/or the second surface is exposed to light having a wavelength within +/−29 nm, within 28 nm, within +/−27 nm, within +/−26 nm, within +/−25 nm, within +/−24 nm, within +/−23 nm, within +/−22 nm, within +/−21 nm, within +/−20 nm, within +/−19 nm, within +/−18 nm, within +/−17 nm, within +/−16 nm, within +/−15 nm, within +/−14 nm, within +/−13 nm, within +/−12 nm, within +/−11 nm, within +/−10 nm, within +/−9 nm, within +/−8 nm, within +/−7 nm, within +/−6 nm, within +/−5 nm, within +/−4 nm, within +/−3 nm, within +/−2 nm or within +/−1 nm of the maximum absorbance wavelength of the fluorophore.
30. Assay according to any of the preceding paragraphs, wherein the first surface and/or the second surface is exposed to light having a wavelength in the range of 200-900 nm.
31. Assay according to paragraph 30, wherein the first and/or the second surface is exposed to light having a wavelength in the range of 295-380 nm, such as in the range of 325-350 nm.
32. Assay according to paragraph 30, wherein the first and/or the second surface is exposed to light having a wavelength in the range of 380-565 nm, such as in the range of 490-535 nm.
33. Assay according to paragraph 30, wherein the first and/or the second surface is exposed to light having a wavelength in the range of 520-590 nm, such as in the range of 550-560 nm.
34. Assay according to paragraph 30, wherein the first and/or the second surface is exposed to light having a wavelength in the range of 535-773 nm, such as in the range of 565-743 nm.
35. Assay according to any of the preceding paragraphs, wherein the light emitted from the fluorophore can be detected by human eye.
36. Assay according to any of the preceding paragraphs, wherein the light emitted from the fluorophore is detected by Assay I or Assay IV.
37. Assay according to any of the preceding paragraphs, wherein the first surface is present on a textile, a dish ware or a hard surface.
38. Assay according to any of the preceding paragraphs, wherein the second surface is present on a textile, a dish ware or a hard surface.
39. Assay according to any of the preceding paragraphs, wherein the light emitted from the fluorophore is invisible to human eye in day light.
40. Method for testing the presence of a fluorophore on a first and/or a second surface comprising the following steps:
   a. Preparing a composition comprising a fluorophore having an absorbance wavelength and an emission wavelength,
   b. applying the composition to the first surface,
   c. submitting the first surface and/or the second surface to a liquid composition,
   d. optionally rinsing and/or drying the first surface and/or the second surface,
   e. exposing the surface and/or the second surface to light having a wavelength within +/−30 nm of the absorbance wavelength of the fluorophore, and
   f. detecting the light emitted from the fluorophore present on the first surface and/or the second surface.
41. Method according to paragraph 40, wherein the fluorophore has an absorbance wavelength in the range of 200-810 nm and an emission wavelength in the range of 200-810 nm, such as the fluorophore has an absorbance wavelength in the range of 224-804 nm and an emission wavelength in the range of 224-804 nm.
42. Method according to any of paragraphs 40-41, wherein the fluorophore has an absorbance wavelength in the range of 320-400 nm, such as in the range of 325-360nm, in the range of 340-360 nm or in the range of 350-360 nm.
43. Method according to any of paragraphs 40-41, wherein the fluorophore has absorbance wavelength in the range of 400-540 nm, such as in the range of 490-535, in the range of 490-495 nm or in the range of 494-535 nm.
44. Method according to any of paragraphs 40-41, wherein the fluorophore has an absorbance wavelength in the range of 550-560, such as in the range of 350-550 nm, in the range of 490-535 nm or in the range of 565-743 nm.
45. Method according to any of paragraphs 40-41, wherein the fluorophore has an absorbance wavelength in the range of 565-810, such as in the range of 565-743 nm.
46. Method according to any of the preceding method paragraphs, wherein the fluorophore is selected from the group consisting of
   a. Xanthene derivatives including fluorescein, rhodamine, Oregon green, eosin and Texas red,
   b. Cyanine derivatives including cyanine, indocarbocyanine, oxacarbocyanine, thiacarbocyanine and merocyanine,
   c. Naphthalene derivatives including dansyl and prodan derivatives,
   d. Coumarin derivatives including hydroxycoumarin, methoxycoumarin and aminocoumarin,
   e. Oxadiazole derivatives, including pyridyloxazole, nitrobenzoxadiazole and benzoxadiazole,
   f. Anthracene derivatives including anthraquinones, including DRAQ5, DRAQ7 and CyTRAK Orange,
   g. Pyrene derivatives including cascade blue,
   h. Oxazine derivatives including Nile red, Nile blue, cresyl violet, oxazine 170,
   i. Acridine derivatives including proflavin, acridine orange, acridine yellow,
   j. Arylmethine derivatives including auramine, crystal violet, malachite green
   k. Tetrapyrrole derivatives including porphin, phthalocyanine, bilirubin and
   l. Anthranilic acid derivatives including N-methylanthraniloyl derivatives formed by reaction of nucleophiles including alcohols and amines with MIA (N-methylisatoic anhydride).
47. Method according to any of the preceding method paragraphs, wherein the fluorophore is selected from the group consisting of anthranilic acid derivatives including N-methylanthraniloyl esters and amides formed by reaction of nucleophiles such as alcohols and amines with MIA (N-methylisatoic anhydride), N-methyl isatoic anhydride (MIA), hydroxycoumarin, methoxycoumarin, Alexa fluor, aminocoumarin, Cy 2, FAM, Alexa fluor 488, Fluorescein FITC, Alexa fluor 430, Alexa fluor 532, HEX, Cy3, TRITC, Alexa fluor 546, Alexa fluor 555, R-phycoerythrin (PE), Rhodamine Red-X, Tamara, Cy3.5 581, Rox, Alexa fluor 568, Red 613, Texas Red, Alexa fluor 594, Alexa fluor 633, Allophycocyanin, Cy5, Alexa fluor 660, Cy5.5, TruRed, Alexa fluor 680, Cy7, DAPI and Hoechst 33258, SYTOX blue, Hoechst 33342, YOYO-1, SYTOX green, TOTO 1 TO-PRO-1, SYTOX orange, Chromomycin A3, Mithamycin, propidium iodide and ethidium bromide.
48. Method according to any of the preceding method paragraphs, wherein the fluorophore is DAPI or N-methylanthraniloyl derivatives formed by reaction of nucleophiles such as alcohols and amines with MIA (N-methylisatoic anhydride).
49. Method according to any of the preceding method paragraphs, wherein the composition comprises organic and/or inorganic material.
50. Method according to paragraph 49, wherein the composition comprises a raw material, a food product, material secreted from human or animal body, particulate material and/or a mixture hereof.
51. Method according to any of paragraphs 49-50, wherein the composition comprises a food product comprising polysaccharides, proteins and/or lipids.
52. Method according to any of paragraphs 49-51, wherein the composition is a food product is selected from the group consisting of mashed potatoes, oatmeal porridge, dairy products and meat products.
53. Method according to any of paragraphs 49-52, wherein the composition comprise a polysaccharide selected from the group consisting of a polysaccharide selected from the group consisting of starch, glycogen, arabinoxylan, cellulose, chitin, pectin, xanthan, carrageenan, arabinogalactan, xyloglucan, xylan, glucuronoxylan, galactan, glucan, arabinan, mannan, glucomannan, galactomannan, xyloglucan, arabinogalactan and pullulan.
54. Method according to any of paragraphs 49-53, wherein the composition comprise a material secreted from human or animal body such as blood, sweat, lipid, grease, sebum, drool, vomit, microorganisms, odor, DNA or mixtures hereof.
55. Method according to any of the preceding method paragraphs, wherein the composition is a soil or a stain.
56. Method according to any of the preceding method paragraphs, wherein the fluorophore is labelled to at least one component in the composition comprising the fluorophore.
57. Method according to any of the preceding method paragraphs, wherein the liquid composition comprises water.
58. Method according to any of the preceding method paragraphs, wherein the liquid composition is a buffered aqueous solution.
59. Method according to any of the preceding method paragraphs, wherein the liquid composition further comprises a surfactant.
60. Method according to any of the preceding method paragraphs, wherein the liquid composition comprises a detergent composition, a dishwashing composition or a composition for cleaning hard surfaces.
61. Method according to any of the preceding method paragraphs, wherein the liquid composition is DMSO or another organic solvent.
62. Method according to any of the preceding method paragraphs, wherein the liquid composition further comprises at least one enzyme.
63. Method according to paragraph 62, wherein the enzyme is selected from the group consisting of hemicellulases, peroxidases, proteases, cellulases, xylanases, lipases, phospholipases, esterases, cutinases, pectinases, mannanases, pectate lyases, keratinases, reductases, oxidases, phenoloxidases, lipoxygenases, ligninases, pullulanases, tannases, pentosanases, malanases, β-glucanases, arabinosidases, hyaluronidase, chondroitinase, laccase, DNase, chlorophyllases, amylases, perhydrolases, peroxidases, xanthanase and mixtures thereof.
64. Method according to any of paragraphs 62-63, wherein the enzyme is an amylase.
65. Method according to any of paragraphs 62-64, wherein the composition comprises an amount of fluorophore that allows the enzyme to degrade or partially degrade the composition.
66. Method according to any of paragraphs 62-65, wherein the composition comprises a polysaccharide comprising at least one fluorophore per 300 monosaccharides, such as at least one fluorophore per 250 monosaccharides, at least one fluorophore per 200 monosaccharides, at least one fluorophore per 150 monosaccharides, at least one fluorophore per 100 monosaccharides or at least one fluorophore per 50 monosaccharides.
67. Method according to any of the preceding method paragraphs, wherein the first surface and/or the second surface is rinsed with water after being submitted to the liquid composition, where the water optionally comprises a fabric conditioner.
68. Method according any of the preceding method paragraphs, wherein the first surface and/or the second surface is exposed to light having a wavelength within +/−29 nm of the absorbance wavelength of the fluorophore, such as within within +/−28 nm, within +/−27 nm, within +/−26 nm, within +/−25 nm, within +/−24 nm, within +/−23 nm, within +/−22 nm, within +/−21 nm, within +/−20 nm, within +/−19 nm, within +/−18 nm, within +/−17 nm, within +/−16 nm, within +/−15 nm, within +/−14 nm, within +/−13 nm, within +/−12 nm, within +/−11 nm, within +/−10 nm, within +/−9 nm, within +/−8 nm, within +/−7 nm, within +/−6 nm, within +/−5 nm, within +/−4 nm, within +/−3 nm, within +/−2 nm or within +/−1 nm of the maximum absorbance wavelength of the fluorophore.
69. Method according any of the preceding method paragraphs, wherein the first and/or the second surface is exposed to light having a wavelength in the range of 200-900 nm.
70. Method according to paragraph 69, wherein the first and/or the second surface is exposed to light having a wavelength in the range of 295-380 nm, such as in the range of 325-350 nm.
71. Method according to paragraph 69, wherein the first and/or the second surface is exposed to light having a wavelength in the range of 380-565 nm, such as in the range of 490-535 nm.
72. Method according to paragraph 69, wherein the first and/or the second surface is exposed to light having a wavelength in the range of 520-590 nm, such as in the range of 550-560 nm.
73. Method according to paragraph 69, wherein the first and/or the second surface is exposed to light having a wavelength in the range of 535-773 nm, such as in the range of 565-743 nm.
74. Method according to any of the preceding method paragraphs, wherein the light emitted from the fluorophore can be detected by human eye.
75. Method according to any of the preceding method paragraphs, wherein the light emitted from the fluorophore is detected by Assay I or Assay IV.
76. Method according to any of the preceding method paragraphs, wherein the first surface is present on a textile, a dish ware or a hard surface.
77. Method according to any of the preceding paragraphs, wherein the second surface is present on a textile, a dish ware or a hard surface.
78. Method according to any of the preceding method paragraphs, wherein the light emitted from the fluorophore is invisible to human eye in day light.
79. Item having a surface, which surface comprises a composition comprising a fluorophore.
80. Item according to paragraph 79, wherein the item is a textile, a dish ware or a hard surface.
81. Item according to any of the preceding item paragraphs, wherein the fluorophore has an absorbance wavelength in the range of 200-810 nm and an emission wavelength in the range of 200-810 nm, such as the fluorophore has an absorbance wavelength in the range of 224-804 nm and an emission wavelength in the range of 224-804 nm.
82. Item according to any of paragraphs 79-81, wherein the fluorophore has an absorbance wavelength in the range of 320-400 nm, such as in the range of 325-360nm, in the range of 340-360 nm or in the range of 350-360 nm.
83. Item according to any of paragraphs 79-81, wherein the fluorophore has absorbance wavelength in the range of 400-540 nm, such as om the range of 490-535, in the range of 490-495 nm or in the range of 494-535 nm.
84. Item according to any of paragraphs 79-81, wherein the fluorophore has an absorbance wavelength in the range of 550-560, such as in the range of 350-550 nm, in the range of 490-535 nm or in the range of 565-743 nm.
85. Item according to any of paragraphs 79-81, wherein the fluorophore has an absorbance wavelength in the range of 565-810, such as in the range of 565-743 nm.
86. Item according to any of the preceding item paragraphs, wherein the fluorophore is selected from the group consisting of
   a) Xanthene derivatives such as fluorescein, rhodamine, Oregon green, eosin and Texas red,
   b) Cyanine derivatives such as cyanine, indocarbocyanine, oxacarbocyanine, thiacarbocyanine and merocyanine,
   c) Naphthalene derivatives such as dansyl and prodan derivatives,
   d) Coumarin derivatives, such as hydroxycoumarin, methoxycoumarin and aminocoumarin,
   e) Oxadiazole derivatives, such as pyridyloxazole, nitrobenzoxadiazole and benzoxadiazole,
   f) Anthracene derivatives such as anthraquinones, including DRAQ5, DRAQ7 and CyTRAK Orange,
   g) Pyrene derivatives such as cascade blue,
   h) Oxazine derivatives such as Nile red, Nile blue, cresyl violet, oxazine 170,
   i) Acridine derivatives such as proflavin, acridine orange, acridine yellow,
   j) Arylmethine derivatives such as auramine, crystal violet, malachite green
   k) Tetrapyrrole derivatives such as porphin, phthalocyanine, bilirubin and
   l) Anthranilic acid derivatives including N-methylanthraniloyl derivatives formed by reaction of nucleophiles including alcohols and amines with MIA (N-methylisatoic anhydride).
87. Item according to any of the preceding item paragraphs, wherein the fluorophore is selected from the group consisting of anthranilic acid derivatives including N-methylanthraniloyl esters and amides formed by reaction of nucleophiles such as alcohols and amines with MIA (N-methylisatoic anhydride), N-methyl isatoic anhydride (MIA), hydroxycoumarin, methoxycoumarin, Alexa fluor, aminocoumarin, Cy 2, FAM, Alexa fluor 488, Fluorescein FITC, Alexa fluor 430, Alexa fluor 532, HEX, Cy3, TRITC, Alexa fluor 546, Alexa fluor 555, R-phycoerythrin (PE), Rhodamine Red-X, Tamara, Cy3.5 581, Rox, Alexa fluor 568, Red 613, Texas Red, Alexa fluor 594, Alexa fluor 633, Allophycocyanin, Cy5, Alexa fluor 660, Cy5.5, TruRed, Alexa fluor 680, Cy7, DAPI and Hoechst 33258, SYTOX blue, Hoechst 33342, YOYO-1, SYTOX green, TOTO 1 TO-PRO-1, SYTOX orange, Chromomycin A3, Mithamycin, propidium iodide and ethidium bromide.
88. Item according to paragraph 87, wherein the fluorophore is DAPI or N-methylanthraniloyl derivatives formed by reaction of nucleophiles such as alcohols and amines with MIA (N-methylisatoic anhydride).
89. Item according to any of the preceding item paragraphs, wherein the composition comprises organic and/or inorganic material.
90. Item according to paragraph 89, wherein the composition comprises raw material, food product, material secreted from human or animal body, particulate material or a mixture hereof.
91. Item according to paragraph 90, wherein the composition comprises a food product comprising polysaccharides, proteins and/or lipids.
92. Item according to any of paragraphs 90-91, wherein the composition is a food product is selected from the group consisting of mashed potatoes, oatmeal porridge, dairy products and meat products.
93. Item according to any of paragraphs 89-92, wherein the composition comprises a polysaccharide selected from the group consisting a polysaccharide selected from the group consisting of starch, glycogen, arabinoxylan, cellulose, chitin, pectin, xanthan, carrageenan, arabinogalactan, xyloglucan, xylan, glucuronoxylan, galactan, glucan, arabinan, mannan, glucomannan, galactomannan, xyloglucan, arabinogalactan and pullulan.
94. Item according to any of paragraphs 89-93, wherein the composition comprises a material secreted from human or animal body such as blood, sweat, lipid, grease, sebum, drool, vomit, microorganisms, odor, DNA or mixtures hereof.
95. Item according to any of the preceding item paragraphs, wherein the composition is a soil or a stain.
96. Item according to any of the preceding item paragraphs, wherein the fluorophore is labelled to at least one component in the composition comprising the fluorophore.
97. Item according to any of the preceding item paragraphs, wherein the item has been washed with a liquid composition comprising water.
98. Item according to paragraph 97, wherein the liquid composition is a buffered aqueous solution.
99. Item according to any of paragraphs 97-98, wherein the liquid composition further comprises a surfactant.
100. Item according to any of paragraphs 97-99, wherein the liquid composition comprises a detergent composition, a dishwashing composition or a composition for cleaning hard surfaces.
101. Item according to any of the preceding item paragraphs, wherein the liquid composition is DMSO or another organic solvent.
102. Item according to any of paragraphs 97-100, wherein the liquid composition further comprises at least one enzyme.
103. Item according to paragraph 102, wherein the enzyme is selected from the group consisting of hemicellulases, peroxidases, proteases, cellulases, xylanases, lipases, phospholipases, esterases, cutinases, pectinases, mannanases, pectate lyases, keratinases, reductases, oxidases, phenoloxidases, lipoxygenases, ligninases, pullulanases, tannases, pentosanases, malanases, β-glucanases, arabinosidases, hyaluronidase, chondroitinase, laccase, DNase, chlorophyllases, amylases, perhydrolases, peroxidases, xanthanase and mixtures thereof.
104. Item according to any of paragraphs 102-103, wherein the enzyme is an amylase.
105. Item according to any of paragraphs 102-104, wherein the composition the composition comprises an amount of fluorophore that allows the enzyme to degrade or partially degrade the composition.
106. Item according to any of paragraphs 102-105, wherein composition comprises a polysaccharide comprising at least one fluorophore per 300 monosaccharides, such as at least one fluorophore per 250 monosaccharides, at least one fluorophore per 200 monosaccharides, at least one fluorophore per 150 monosaccharides, at least one fluorophore per 100 monosaccharides or at least one fluorophore per 50 monosaccharides.
107. Composition comprising a fluorophore having an absorbance wavelength in the range of 200-810 nm and an emission wavelength in the range of 200-810 nm.
108. Composition according to paragraph 107, wherein the fluorophore has an absorbance wavelength in the range of 224-804 nm and an emission wavelength in the range of 224-804 nm.
109. Composition according to paragraph 108, wherein the fluorophore has an absorbance wavelength in the range of 320-400 nm, such as in the range of 325-360nm, in the range of 340-360 nm or in the range of 350-360 nm.
110. Composition according to paragraph 108, wherein the fluorophore has absorbance wavelength in the range of 400-540 nm, such as om the range of 490-535, in the range of 490-495 nm or in the range of 494-535 nm.
111. Composition according to paragraph 108, wherein the fluorophore has an absorbance wavelength in the range of 550-560, such as in the range of 350-550 nm, in the range of 490-535 nm or in the range of 565-743 nm.
112. Composition according to paragraph 108, wherein the fluorophore has an absorbance wavelength in the range of 565-810, such as in the range of 565-743 nm.
113. Composition according to any of the preceding composition paragraphs, wherein the fluorophore is selected from the group consisting of
   a) Xanthene derivatives including fluorescein, rhodamine, Oregon green, eosin and Texas red,
   b) Cyanine derivatives including cyanine, indocarbocyanine, oxacarbocyanine, thiacarbocyanine and merocyanine,
   c) Naphthalene derivatives including dansyl and prodan derivatives,
   d) Coumarin derivatives, including hydroxycoumarin, methoxycoumarin and aminocoumarin,
   e) Oxadiazole derivatives, including pyridyloxazole, nitrobenzoxadiazole and benzoxadiazole,
   f) Anthracene derivatives including anthraquinones, including DRAQ5, DRAQ7 and CyTRAK Orange,
   g) Pyrene derivatives including cascade blue,
   h) Oxazine derivatives including Nile red, Nile blue, cresyl violet, oxazine 170,
   i) Acridine derivatives including proflavin, acridine orange, acridine yellow,
   j) Arylmethine derivatives including auramine, crystal violet, malachite green
   k) Tetrapyrrole derivatives including porphin, phthalocyanine, bilirubin and
   l) Anthranilic acid derivatives including N-methylanthraniloyl derivatives formed by reaction of nucleophiles including alcohols and amines with MIA (N-methylisatoic anhydride)
114. Composition according to any of the preceding composition paragraphs, wherein the fluorophore is selected from the group consisting of anthranilic acid derivatives including N-methylanthraniloyl esters and amides formed by reaction of nucleophiles such as alcohols and amines with MIA (N-methylisatoic anhydride), N-methyl isatoic anhydride (MIA), hydroxycoumarin, methoxycoumarin, Alexa fluor, aminocoumarin, Cy 2, FAM, Alexa fluor 488, Fluorescein FITC, Alexa fluor 430, Alexa fluor 532, HEX, Cy3, TRITC, Alexa fluor 546, Alexa fluor 555, R-phycoerythrin (PE), Rhodamine Red-X, Tamara, Cy3.5 581, Rox, Alexa fluor 568, Red 613, Texas Red, Alexa fluor 594, Alexa fluor 633, Allophycocyanin, Cy5, Alexa fluor 660, Cy5.5, TruRed, Alexa fluor 680, Cy7, DAPI and Hoechst 33258, SYTOX blue, Hoechst 33342, YOYO-1, SYTOX green, TOTO 1 TO-PRO-1, SYTOX orange, Chromomycin A3, Mithamycin, propidium iodide and ethidium bromide.
115. Composition according to paragraph 114, wherein the fluorophore is DAPI or N-methylanthraniloyl derivatives formed by reaction of nucleophiles such as alcohols and amines with MIA (N-methylisatoic anhydride).
116. Composition according to any of the preceding composition paragraphs, wherein the composition comprises organic and/or inorganic material.
117. Composition according to paragraph 116, wherein the composition comprises a raw material, a food product, material secreted from human or animal body, particulate material and/or a mixture hereof.
118. Composition according to any of paragraphs 116-117, wherein the composition comprises a food product comprising polysaccharides, proteins and/or lipids.
119. Composition according to any of paragraphs 116-118, wherein the composition is a food product is selected from the group consisting of mashed potatoes, oatmeal porridge, dairy products and meat products.
120. Composition according to any of paragraphs 116-119, wherein the composition comprise a polysaccharide selected from the group consisting of a polysaccharide selected from the group consisting of starch, glycogen, arabinoxylan, cellulose, chitin, pectin, xanthan, carrageenan, arabinogalactan, xyloglucan, xylan, glucuronoxylan, galactan, glucan, arabinan, mannan, glucomannan, galactomannan, xyloglucan, arabinogalactan and pullulan.
121. Composition according to any of paragraphs 116-120, wherein the composition comprise a material secreted from human or animal body such as blood, sweat, lipid, grease, sebum, drool, vomit, cells, microorganisms, odor, DNA or mixtures hereof.
122. Composition according to any of the preceding composition paragraphs, wherein the composition is a soil or a stain.
123. Composition according to any of the preceding composition paragraphs, wherein the fluorophore is labelled to at least one component in the composition comprising the fluorophore.
124. An assay according to any of the preceding assay paragraphs, comprising the following steps:
   a. Preparing a soil comprising a fluorophore having an absorbance wavelength and an emission wavelength,
   b. applying the soil to the first surface,
   c. submitting the first surface and/or the second surface to a wash liquor optionally comprising at least one enzyme,
   d. optionally rinsing and/or drying the first surface and/or the second surface,
   e. exposing the surface and/or the second surface to light having a wavelength within +/−30 nm of the absorbance wavelength of the fluorophore, and
   f. detecting the light emitted from the fluorophore present on the first surface and/or the second surface.
125. A method according to any of the preceding method paragraphs, comprising the following steps:
   a. Preparing a soil comprising a fluorophore having an absorbance wavelength and an emission wavelength,
   b. Applying the soil to the first surface,
   c. submitting the first surface and/or the second surface to a wash liquor optionally comprising at least one enzyme,
   d. Optionally rinsing and/or drying the first surface and/or the second surface,
   e. exposing the surface and/or the second surface to light having a wavelength within +/−30 nm of the absorbance wavelength of the fluorophore, and
   f. detecting the light emitted from the fluorophore present on the first surface and/or the second surface.

### Assays

### Composition of Model Detergent A (Liquid)

Ingredients: 12% LAS, 11% AEO Biosoft N25-7 (NI), 7% AEOS (SLES), 6% MPG (monopropylene glycol), 3% ethanol, 3% TEA, 2.75% cocoa soap, 2.75% soya soap, 2% glycerol, 2% sodium hydroxide, 2% sodium citrate, 1% sodium formiate, 0.2% DTMPA and 0.2% PCA (all percentages are w/w)

### Assay I

The presence of a fluorophore can be quantified by means of the brightness of the surface (as a measure of the observed fluorescence) when illuminated with UV light. The brightness can be expressed as the intensity of the light reflected from the surface when illuminated with UV light. The intensity of the light reflected from surfaces containing higher amounts of the composition comprising a fluorophore will be higher than for surfaces containing less or no composition comprising a fluorophore. Therefore the intensity of the reflected light can be used to measure the presence of a fluorophore. Intensity measurements were conducted with a DigiEye (VeriVide) in UV mode (for example with a wavelength of 375 nm) equipped with a Nikon D90 camera, which was used to capture an image of the surface placed on a white background inside the DigiEye. In order to extract values for the light intensity from the scanned images, 24-bit pixel values from the images were converted into values for red, green and blue (RGB). The intensity value (Int) was calculated by adding the RGB values together as vectors and then taking the length of the resulting vector: Int=Square root(r²+g²+b²). The presence of fluorophore can be evaluated by measuring the intensity of the surface using the DigiEye and can be expressed as the delta intensity value (Delta Int) for example compared to unwashed or washed textile (dishware or hard surfaces) still containing the fluorophore. The larger the negative Delta Int, the lower presence of fluorophore. Different types of surfaces show different background intensities depending on the type of surface (e.g. different types of textiles etc.)

### Assay II

### Washing Test in Beaker

500 ml deionized water was added in each beaker. The water was heated to 40° C. and a liquid composition (e.g. wash liquor) was added and stirred for 5 minutes. If an enzyme should be included in the testing, the enzyme was added into one beaker together with the liquid composition. 6 swatches were added into each beaker, and stirred for 15 minutes. The swatches were rinsed in tap water, and results evaluated.

### Assay III

### Terg-O-Tometer (TOM) Wash Assay

The Tergo-To-Meter (TOM) is a medium scale model wash system that can be applied to test 12 different wash conditions simultaneously. A TOM is basically a large temperature controlled water bath with up to 12 open metal beakers submerged into it. Each beaker constitutes one small top loader style washing machine and during an experiment, each of them will contain a solution of a specific detergent/enzyme system and the soiled and unsoiled fabrics its performance is tested on. Mechanical stress is achieved by a rotating stirring arm, which stirs the liquid within each beaker. Because the TOM beakers have no lid, it is possible to withdraw samples during a TOM experiment and assay for information on-line during wash.

The TOM model wash system is mainly used in medium scale testing of detergents and enzymes at US or LA/AP wash conditions. In a TOM experiment, factors such as the ballast to soil ratio and the fabric to wash liquor ratio can be varied. Therefore, the TOM provides the link between small scale experiments, such as AMSA and mini-wash, and the more time consuming full scale experiments in top loader washing machines.

Equipment: The water bath with 12 steel beakers and 1 rotating arm per beaker with capacity of 500 or 1200 mL of detergent solution. Temperature ranges from 5 to 80° C. The water bath has to be filled up with deionised water. Rotational speed can be set up to 70 to 120 rpm/min.

Set temperature in the Terg-O-Tometer and start the rotation in the water bath. Wait for the temperature to adjust (tolerance is +/−0.5° C.)

All beakers shall be clean and without traces of prior test material.

Prepare wash solution with desired amount of detergent, temperature and water hardness in a bucket. Let detergent dissolve during magnet stirring for 10 min. Wash solution shall be used within 30 to 60 min after preparation.

Add 1000 ml wash solution into a TOM beaker

Start agitation at 60 rpm and optionally add enzymes to the beaker.

Sprinkle the swatches into the beaker and then the ballast load.

Time measurement start when the swatches and ballast are added to the beaker.

Wash for 15 minutes

Stop agitation

Transfer the wash load from TOM beaker to a sieve and rinse with cold tap water for 5 minutes, rinse swatches washed with or without enzyme separately. Press gently the water out by hand and place the test swatches on a tray covered with a paper. Add another paper on top of the swatches. Let the swatches dry overnight.

### Assay IV

After being washed and rinsed, the swatches were spread out flat and allowed to air dry at room temperature overnight. All washes are evaluated the day after the wash. Light reflectance evaluations of the swatches were done using a Macbeth Color Eye 7000 reflectance spectrophotometer with very small aperture. The measurements were made without UV in the incident light and remission at 460 nm was extracted.

### Assay V

### Full Scale Wash:

This is the test method used to test under full scale wash under EU conditions, where real laundry items can be washed. The real items (e.g. Shirts) are added to each wash together with a liquid composition e.g. a detergent and optionally comprising an enzyme. The enzymes are added on basis of weight percent of the detergent dosage in each wash. After wash, the real items are dried overnight.

### Equipment Used:

Washing machine: Miele Softtronic W2445
Water meters and automatically data collection system

### For The Preparation and Adjustment of Water Hardness the Following Ingredients are Needed:

Calcium chloride (CaCl₂.2H₂O)
Magnesium chloride (MgCL₂.6H₂O)
Sodium Hydrogen Carbonate (NaHCO₃)

### Wash Conditions

Temperature: 40° C.
Washing programme: Normal cotton wash without pre-wash: “Cottons”.
Water level 12-13 L with “water plus”
Water hardness: Standard EU conditions: 15° dH, Ca2+:Mg2+:HCO3=4:1:7.5

### Detailed Steps to Carry Out Full Scale Wash Trial

1. Select wash program as in study plan.
2. The detergent and Enzyme are placed in the wash drum in a “washing ball” (both liquid and powder detergents). Place it at the bottom.
3. Place the real items in the wash drum.
4. Start digital water meter
5. Start the washer by pressing the knob START
6. After wash, take out real items and dry them at the room temperature overnight.

### Drying Procedure

Hang the items in line and dry at room temperature overnight.

### EXAMPLES

### Chemicals:

EtOH: 96% EtOH was used unless otherwise stated.
Water: MQ water was used unless otherwise stated.

### Example 1

Preparing a Composition (Rice Starch) Labelled with a Fluorophore

1 g of rice starch (supplied by Fluka) was suspended in 100 mL of borate buffer (100 mM, pH 8), heated to 100° C. while stirring for 20 minutes to invoke gelatinization followed by cooling to room temperature and diluted with 100 mL of DMSO (dimethylsulfoxide) (the solution became warm). The solution was cooled to room temperature followed by addition of 8 mL of freshly prepared MIA solution (fluorophore) in DMSO (N-methyl isatoic anhydride, 10 mg/mL, 400 μg MIA/5 mg sugar). The mixture was stirred at room temperature for 50 minutes followed by precipitation of the labelled starch with two V/V equivalents of ethanol, centrifugation at 8500 rpm for 15 minutes, removal of the supernatant and redissolution of the solid residue in 250 mL of water. The solution was then heated to 100° C. for 10 minutes (gelatinization) followed by precipitation with two equivalents of ethanol (V/V), centrifugation at 8500 rpm for 15 minutes, removal of the supernatant and drying of the solid residue in the freeze dryer (Coolsafe CS110-4 pro freeze dryer (Scanvac)) overnight. This procedure gave 586 mg powder fluorescence labelled rice starch as a white powder.

The powder was suspended in MQ water to obtain a 2.5% (25 mg/mL) solution. The solution was heated to 100° C. for 20 min to gelatinize the starch and make it homogeneous. This composition can be applied to a surface such as a surface on textile dishware or hard surfaces using standard techniques for example by applying to textile as a warm solution.

### Example 2

Preparing a Composition (Brevundimonas Biofilm (DNA)) Labelled with a Fluorophore

### Isolating Laundry Specific Bacterial Strains

One strain of Brevundimonas sp. isolated from laundry was used in the present example.

The Brevundimonas sp. was isolated during a study, where the bacterial diversity in laundry after washing at 15, 40 and 60° C., respectively, was investigated. The study was conducted on laundry collected from Danish households. For each wash, 20 g of laundry items (tea towel, towel, dish cloth, bib, T-shirt armpit, T-shirt collar, socks) in the range 4:3:2:2:1:1:1 was used. Washing was performed in a Laundr-O-Meter (LOM) at 15, 40 or 60° C. For washing at 15 and 40° C., Ariel Sensitive White & Color was used, whereas WFK IEC-A* model detergent was used for washing at 60° C. Ariel Sensitive White & Color was prepared by weighing out 5.1 g and adding tap water up to 1000 ml followed by stirring for 5 minutes. WFK IEC-A* model detergent (which is available from WFK Testgewebe GmbH) was prepared by weighing out 5 g and adding tap water up to 1300 ml followed by stirring for 15 min. Washing was performed for 1 hour at 15, 40 and 60° C., respectively, followed by 2 times rinsing with tap water for 20 min at 15° C.

Laundry was sampled immediately after washing at 15, 40 and 60° C., respectively. Twenty grams of laundry was added 0.9% (w/v) NaCI (1.06404; Merck, Damstadt, Germany) with 0.5% (w/w) tween 80 to yield a 1:10 dilution in stomacher bag. The mixture was homogenized using a Stomacher for 2 minutes at medium speed. After homogenization, ten-fold dilutions were prepared in 0.9% (w/v) NaCI. Bacteria were enumerated on Tryptone Soya Agar (TSA) (CM0129, Oxoid, Basingstoke, Hampshire, UK) incubated aerobically at 30° C. for 5-7 days. To suppress growth of yeast and moulds, 0.2% sorbic acid (359769, Sigma) and 0.1% cycloheximide (18079; Sigma) were added. Bacterial colonies were selected from countable plates and purified by restreaking twice on TSA. For long time storage, purified isolates were stored at -80° C. in TSB containing 20% (w/v) glycerol (49779; Sigma).

### Preparation of Brevundimonas Biofilm Swatch

Brevundimonas was inoculated in 6×10 ml TSB and incubated for approximately 20 hrs.

The cultures were pooled and spinned down at 3000 G in 7 min. Afterwards the pellet was resuspended in 20 mL 50% TSB.

Two dilutions of the resuspended cultures were made:

A 10× dilution was made by adding 100 μL bacterial suspension to 900 μL 50% TSB

A 20× dilution was made by adding 400 μL of the 10× dilution to 400 μL 50% TSB (See Table below)

| | 1 | 2 | 3 |
|---|---|---|---|
| A | Blank: 100 μl TSB 50% | Biofilm: 100 μl 10x | Biofilm: 100 μl 20x |
| | | dilution | dilution |
| B | Blank: 100 μl TSB 50% | Biofilm: 100 μl 10x | Biofilm: 100 μl 10x |
| | | dilution | dilution |
| C | Blank: 100 μl TSB 50% | Biofilm: 100 μl 10x | Biofilm: 100 μl 10x |
| | | dilution | dilution |
| D | Blank: 100 μl TSB 50% | Biofilm: 100 μl 10x | Biofilm: 100 μl 10x |
| | | dilution | dilution |

The absorbance at 600 nm (OD₆₀₀) was measured on a spectrophotometer (Polarstar Omega, BMG labtech). Based on the spectrophotometric measurements as indicated above the resuspended biofilm was diluted to an OD₆₀₀=0.03 in 50% TSB.

1 sterile 5×5 swatch was placed in a petri dish and 20 mL diluted resuspended biofilm was added onto the swatch. The petri dish containing the swatch was placed on a shaking plate (75 rpm) and incubated for approximately 24 hours in a climate chamber at 15° C.

The supernatant was removed from the petri dish, followed by two times rinsing with 20 mL 0.9% NaCI solution.

### Fluorescence Labeling of Brevundimonas Biofilm Swatch

Brevundimonas biofilm contains DNA which can be visualized with a DAPI fluorophore (4′,6′-diamidino-2-phenylindole). The DAPI stain binds non-covalently in the minor groove of dsDNA, preferentially binding to AT-base clusters. DAPI is a fluorophore that exhibits blue fluorescence and binding to DNA results is an approximate 20-fold enhancement in fluorescence. The fluorescence can be measured by excitation at 340-360 nm and detecting fluorescence at 440-460 nm. For the visualization with DAPI, a DAPI stock solution: 20 mg/mL was prepared. 20 μl of the DAPI stock solution was diluted in 40 mL phosphate buffer pH 8 to give a 10 μg/mL solution of DAPI.

A sample of the Brevundimonas biofilm swatch was taken (sample size: 1.5 cm in diameter) and placed in a microtiter plate together with 1 mL of the 10 μg/mL DAPI solution. The swatch was incubated at 37° C. in the dark on a shaking plate (350 rpm/5 min). After incubation the supernatant was removed and the swatch was washed with 1 mL milli Q water (300 rpm/5 min). The liquid was removed and the swatch was dried on a filter paper for a few minutes.

Detection of fluorescence was performed by exciting the labeled DNA at 340-360 nm or 365 nm and the fluorescence emission was detected around 440-460 nm or by human eye as described in example 8 below.

### Example 3

Preparing a Composition (Carrageenan) Labelled with a Fluorophore

2 g of carrageenan (Sigma) was dissolved in 200 mL of borate buffer (100 mM, pH 8) at room temperature followed by addition of 200 mL of DMSO and stirring at room temperature for 1 h. The solution was added 20 mL of freshly prepared MIA solution in DMSO (N-methyl isatoic anhydride, 10 mg/mL, 500 μg MIA/5 mg sugar). The mixture was stirred at room temperature for 40 minutes followed by precipitation of the labelled carrageenan as a gel with 1.5 V/V equivalents of ethanol and standing for 30 min. The gel was physically removed from the liquid and pressed to remove excess liquid. The gel was redissolved in 200 mL of water followed by precipitation of the labeled carrageenan as a gel by addition of 7.5 V/V equivalents of ethanol and standing for 3 h. The mixture was centrifuged (3000 rpm, 20 min) and the supernatant was removed followed by freeze-drying (Coolsafe CS110-4 pro freeze dryer (Scanvac)) of the precipitated carrageenan gel. This gave 2.07 g of fluorescence labeled carrageenan as a white powder. Visualization of fluorescence was performed by exciting the labeled polysaccharide at for example 365 nm and detecting emission of fluorescence at 460 nm or by human eye as described in example 8 below.

### Example 4

Preparing a Composition (Destarched Maize) Labelled with a Fluorophore

20.1 g of destarched maize (the maize was supplied by Bioteknologisk Intitut, Aalborg University, Kolding and was subsequently destarched at Novozymes by treatment with amylases) was suspended in 1.5 L of borate buffer (100 mM, pH 8) and 4.5 L of DMSO at room temperature (suspension of insoluble material) (the suspension became warm upon addition of DMSO and buffer). After cooling to room temperature 186 mL of freshly prepared MIA solution in DMSO (N-methyl isatoic anhydride, 10 mg/mL, 465 μg MIA/5 mg maize). The mixture was stirred at room temperature for 45 minutes followed by centrifugation (3500 rpm, 4 min) and removal of the supernatant. The solid labeled destarched maize was then washed by adding 480 mL water to the solids, vigorous shaking, centrifugation (3500 rpm, 4 min) and removal of the supernatant. This procedure was repeated three times with 480 mL of 1:1 water/99% EtOH and one additional time with 480 mL 99% EtOH. The produced solid was freeze-dried (Coolsafe CS110-4 pro freeze dryer (Scanvac)) and grinded to produce 16.3 g fluorescence labeled destarched maize as a light brown solid.

This composition can be applied to a surface such as a surface on textile, dishware or hard surfaces using standard techniques for example by applying to textile as a warm solution/suspension.

### Example 5

Preparing a Composition (Mashed Potato) Labelled with a Fluorophore

400 g of potato (Estima) was added to 500 ml of tab water and heated until 100° C. The potatoes were cooked for 30 min. The water was drained from the cooked potatoes and the potatoes were then mashed with a potato masher until there were no lumps left. 10 g mashed potato was suspended in 100 mL of borate buffer (100 mM, pH8), heated to 100° C. while stirring for 30 minutes. The mashed potatoes was cooled down to room temperature and diluted with 100 ml of DMSO. Cooled to room temperature, and added 6 mL of freshly prepared MIA/DMSO solution (10 mg MIA/1 mL DMSO). Stirred at room temperature for 50 minutes. Added 2 V/V equivalents of ethanol, centrifugation (8500 rpm, 15 minutes), removal of the supernatant. The solid was re-dissolved in 250 ml of water and heated to 100° C. for 20 minutes. Cooled to room temperature, and added 2 V/V equivalents of ethanol, centrifugation (8500 rpm, 10 minutes), and removal of the supernatant. The solid residue was freeze-dried (Coolsafe CS110-4 pro freeze dryer (Scanvac)) overnight.

The freeze dryed solid residue was suspended in MQ water to obtain a 2.5% (25 mg/mL) solution. The solution was heated to 100° C. for 20 min to gelatinize the starch and make it homogeneous. This material can be applied to surfaces on textile, dishware or hard surfaces using standard techniques for example by applying to textile as a warm solution.

### Example 6

Preparing a Composition (Oatmeal Porridge) Labelled with a Fluorophore

3 g porridge oats (supplied by Quaker) was suspended in 100 mL of borate buffer (100 mM, pH8), heated to 100° C. while stirring for 30 minutes. Cooled down to room temperature and diluted with 100 ml of DMSO. Cooled to room temperature, and added 9 mL of freshly prepared MIA/DMSO solution (10 mg/ml). Stirred at room temperature for 50 minutes. Added 2 equivalents of ethanol, centrifugation (8500 rpm, 15 minutes), removal of the supernatant. Re-dissolved the solid residue in 250 ml of water, and heated to 100° C. for 20 minutes. Cooled to room temperature, and added 2 equivalents of ethanol, centrifugation (8500 rpm, 10 minutes), and removal of the supernatant. The solid residue was freeze-dried (Coolsafe CS110-4 pro freeze dryer (Scanvac)) overnight. 1 g labelled porridge oats was dissolved in 40 ml deionized water, the solution (2.5%) was heated to 100° C. for 20 minutes, and applied 200 μl to surfaces on textile, dishware or hard surfaces.

### Example 7

Preparing a Composition (Milk) Labelled with a Fluorophore

CBS-X was dissolved in fresh milk at concentration of 0.0084% weight percentage, and stirred for 10 min to ensure completely dissolution. 200 μL of OB labelled milk was pipetted on to polyester textile (size 5*5 cm, knitted polyester), a dishware or a hard surface. The composition was dried overnight at room temperature.

### Example 8

Testing a Composition (Mashed Potato & Porridge Oats) Labelled with a Fluorophore in a Assay II

The compositions of example 5 & 6 were prepared and 200 μl was applied to a textile swatch (Polyester/Cotton 65/35%, PCN-01, CFT, Center For Testmaterials, the Netherlands). Thus, making swatches with fluorescence labeled mashed potato and porridge oats stains.

The swatches were then washed according to Assay II and according to the following table:

| Wash 1 | Wash 2 |
|---|---|
| Wash liquor comprising | Wash liquor comprising model detergent A |
| model detergent A 3.3 g/L | 3.3 g/L + 5.05 mg enzyme protein/L of SEQ |
| | ID NO: 1 |

SEQ ID NO: 1 is a protein having alpha-amylase activity.

### Evaluation and Results

The fluorescence labeled mashed potato & porridge oats stains were evaluated by the human eye in daylight and when using a UV flash light (300 mW, 365 nm) as light source. A trained test person evaluated the presence of composition left on the swatch in daylight and detected the fluorescence signal emitted from the swatch and ranked it according to the following scale:

Scale from A-E, A=Strong fluorescence, B=medium fluorescence, C=low fluorescence, D=trace fluorescence and E=no detectable fluorescence.

The following results were obtained:

| | | | Fluorescense |
|---|---|---|---|
| Composition | Wash condition | Day light | with UV light |
| Mashed Potato | Wash 1: without | Not visible | A |
| | enzyme | composition left | |
| Mashed Potato | Wash 2: with | Not visible | E |
| | enzyme | composition left | |
| Oatmeal | Wash 1: without | Not visible | A |
| | enzyme | composition left | |
| Oatmeal | Wash 2: with | Not visible | D |
| | enzyme | composition left | |

The results show that the composition left on the swatch was not visible after wash. However, when exposed to UV-light, the composition left on the swatch became visible. Washing with enzyme removed more composition from the swatch.

### Example 9

Testing a Composition (Rice Starch) Labelled with a Fluorophore in Assay III

The compositions of example 1, 1 g labelled starch was prepared and 200 μl was applied to a textile swatch (Polyester/Cotton 65/35% of PCN-01)

The swatches were then washed according to Assay III and according to the following table:

| Wash 1 | Wash 2 |
|---|---|
| Wash liquor comprising | Wash liquor comprising model detergent A |
| model detergent A 3.3 g/L | 3.3 g/L + 5.05 mg enzyme protein/L of SEQ |
| | ID NO: 1 |

### Evaluation and Results

The fluorescence labeled rice starch stains were measured by using a Digieye as described in Assay I.

The following results were obtained:

| | | | Fluorescense |
|---|---|---|---|
| Composition | Wash condition | Day light | with UV light |
| rice starch | Wash 1: Without | Not visible | A |
| | enzyme | composition left | |
| rice starch | Wash 2: with | Not visible | E |
| | enzyme | composition left | |

The results show that the composition left on the swatch was not visible after wash. However, when exposed to UV-light, the composition left on the swatch became visible. Washing with enzyme removed more composition from the swatch.

### Example 10

Testing a Composition (Rice Starch, Mashed Potato and Porridge Oats) Labelled with a Fluorophore in Assay V

The compositions of 1, 5 and 6 were prepared 200 μl applied to a Shirt

The swatches were then washed according to Assay V and according to the following table:

| Wash 1 | Wash 2 |
|---|---|
| Wash liquor comprising | Wash liquor comprising model detergent A |
| model detergent A 3.3 g/L | 3.3 g/L + 0.42 mg enzyme protein/L of SEQ |
| | ID NO: 1 |

Evaluation and Results

The fluorescence labeled rice starch, mashed potato and porridge oats stains were measured by using a Digieye as described in Assay I.

The following results were obtained:

| Composition | Wash condition | R | G | B | Int | Delta Int |
|---|---|---|---|---|---|---|
| Oatmeal | Wash 1 | 9 | 11 | 24 | 27.9 | 0.0 |
| Oatmeal | Wash 2 | 4 | 5 | 7 | 9.5 | −18.4 |
| Clean Textile - wowen shirt | | 4 | 5 | 7 | 9.5 | |
| 100% polyester, H&M | | | | | | |
| rice starch | Wash 1 | 7 | 9 | 17 | 20.5 | 0.0 |
| rice starch | Wash 2 | 4 | 5 | 7 | 9.5 | −11.0 |
| Clean Textile - wowen shirt | | 5 | 5 | 7 | 9.9 | |
| 100% polyester, H&M | | | | | | |
| Mashed Potato | Wash 1 | 8 | 10 | 22 | 25.5 | 0.0 |
| Mashed Potato | Wash 2 | 6 | 7 | 12 | 15.1 | −10.3 |

| | | | | |
|---|---|---|---|---|
| Clean Textile - wowen shirt | 6 | 7 | 12 | 15.1 |
| 100% polyester, H&M | | | | |

The results show that the composition left on the swatch was significantly reduced when washed in the prescence of a amylase (Wash 2) as compared to wash in the absence of an amylase (Wash 1).

### Example 11

Preparing and Testing a Composition (Salmon Sperm DNA) Labelled with a Fluorophore (Propidium Iodid and SYBR-Safe)

The present example describes a method for testing the presence of DNA in textile.

Dilutions of Salmon Sperm DNA (D1626, Sigma) were prepared and 0, 0.060, 0.125, 0.190, 0.250 and 320 mg DNA/cm² was added to polyester swatches (WFK30A) (2 cm in diameter). Swatches were dried overnight in a fumehood. DNA in swatches was quantified by use of the fluorophores propidium iodid and SYBR-safe. Hundred microliter of Propidium iodid (0.03M) (P3566, Invitrogen) and SYBR-safe (two drops in 100 ml of water) (P11496, Molecular probes), respectively, was added to swatches with DNA. Swatches were dried overnight in a fume hood.

After drying, the emission of light from the fluorophore was tested in a UV chamber and quantified by using fluorometer. Propidium iodide was quantified at absorbance wavelength 535 nm and emission wavelength 617 nm, whereas SYBR-safe was quantified at absorbance wavelength 502 nm and emission wavelength 530 nm.

| DNA conc. | Emitted light intensity | Emitted light intensity |
|---|---|---|
| (mg/cm²) | (Propidium iodide) | (SYBR-safe)) |
| 0.320 | 1411869 | — |
| 0.250 | 1326804 | 611444 |
| 0.190 | 813119 | 452475 |
| 0.125 | 832291 | 300682 |
| 0.060 | 687887 | 223798 |
| 0 | 32793 | 164004 |
| | R² = 0.89 | R² = 0.96 |

### Example 12

Preparing and Testing a Composition (Starch) Labelled with a Visible Fluorophore (DTAF)

50 mg of rice starch (supplied by Fluka), mashed potato (Estima), or oatmeal (Quaker) was suspended in 5 mL of carbonate buffer (100 mM, pH 9.5), heated to 100° C. while stirring for 20 minutes to invoke gelatinization followed by cooling to room temperature. Fluorophore (3.33 mg) was dissolved into 5 mL carbonate buffer (100 mM, pH 9.5) and added to the starch solution. The mixture was stirred at room temperature overnight. The solution was purified by precipitation using two equivlaents of isopropylalcohol (20 mL). The precipitate was separated by centrifugation at 8500 rpm for 15 minutes and the supernatant was removed. The pellet was dissolved in 5 mL of water and heated to 100° C. for 10 minutes (gelatinization) followed by precipitation with two equivalents of isopropylalcohol (10 mL), centrifugation at 8500 rpm for 15 minutes, removal of the supernatant and drying of the solid residue in the freeze dryer (Coolsafe CS110-4 pro freeze dryer (Scanvac)) overnight.

The powder was suspended in MQ water to obtain a 2.5% (25 mg/mL) solution. The solution was heated to 100° C. for 20 min to gelatinize the starch and make it homogeneous. This composition can be applied to a surface such as a surface on textile dishware or hard surfaces using standard techniques for example by applying to textile as a warm solution.

Testing a Composition (Starch) Labelled with a Fluorophore (DTAF)

The compositions of fluorescently labelled starch was prepared and 20 μl was applied to a textile swatch (Cotton 100% of WFK-10A)

The swatches were then washed according to Assay III according to the following table:

| Wash 1 | Wash 2 |
|---|---|
| Wash liquor comprising | Wash liquor comprising model detergent A |
| model detergent A 3.3 g/L | 3.3 g/L + 0.5 mg enzyme protein/L of of |
| | SEQ ID NO: 1 |

### Evaluation and Results

The fluorescence labeled rice starch, mashed potato, and oatmeals stains were measured by using a Digieye as described in Assay I.

Scale from A-E, A=Strong fluorescence, B=medium fluorescence, C=low fluorescence, D=trace fluorescence and E=no detectable fluorescence The following results were obtained

| | | | Fluorescense |
|---|---|---|---|
| Composition | Wash condition | Day light | with UV light |
| rice starch- | Wash 1: Without | Yellow stain left | A |
| DTAF | enzyme | on textile | |
| rice starch- | Wash 2: with | No visible | E |
| DTAF | enzyme | composition left | |
| Mashed Potato- | Wash 1: without | Yellow stain left | A |
| DTAF | enzyme | on textile | |
| Mashed Potato- | Wash 2: with | No visible | E |
| DTAF | enzyme | composition left | |
| Oatmeal-DTAF | Wash 1: without | Yellow stain left | A |
| | enzyme | on textile | |
| Oatmeal-DTAF | Wash 2: with | No visible | D |
| | enzyme | composition left | |

The results show that the composition labelled with DTAF left on the swatch after a wash without enzyme showed a yellow stain in visible light and green fluorescence with UV light. Washing with enzyme removed the composition from the swatch in both visible and UV light.

### Example 13

Preparing and Testing a Composition (Galactomannan) Labelled with a Fluorophore (MIA)

50 mg of galactomannan (guar gum, supplied by Sigma) was suspended in 5 mL of carbonate buffer (100 mM, pH 9.5), heated to 100° C. while stirring for 20 minutes to invoke gelatinization followed by cooling to room temperature. DTAF fluorophore (3.33 mg) was dissolved into 5 mL carbonate buffer (100 mM, pH 9.5) and added to the guar gum solution. The mixture was stirred at room temperature overnight. The solution was purified by precipitation using two equivlaents of isopropylalcohol (20 mL). The precipitate was separated by centrifugation at 8500 rpm for 15 minutes and the supernatant was removed. The pellet was dissolved in 5 mL of water and heated to 100° C. for 10 minutes (gelatinization) followed by precipitation with two equivalents of isopropylalcohol (10 mL), centrifugation at 8500 rpm for 15 minutes, removal of the supernatant and drying of the solid residue in the freeze dryer (Coolsafe CS110-4 pro freeze dryer (Scanvac)) overnight.

The powder was suspended in MQ water to obtain a 1.0% (10 mg/mL) solution. The solution was heated to 100° C. for 20 min to gelatinize the gum and make it homogeneous. This composition can be applied to a surface such as a surface on textile dishware or hard surfaces using standard techniques for example by applying to textile as a warm solution.

Testing a Composition (Galactomnannan) Labelled with a Fluorophore

The compositions of labelled galactomannan was prepared and 50μl was applied to a textile swatch (Cotton 100% of WFK-10A).

The swatches were then washed according to Assay III, using a powder detergent for a wash solution over pH 10, according to the following table:

| Wash 1 | Wash 2 |
|---|---|
| Wash liquor comprising | Wash liquor comprising model detergent A |
| model detergent A 3.3 g/L | 3.3 g/L + 0.4 mg enzyme protein/L of |
| | MannanaseSEQ ID NO: 2. |

### Evaluation and Results

The fluorescence labeled galactomannan stains were measured by using a Digieye as described in Assay I.

Scale from A-E, A=Strong fluorescence, B=medium fluorescence, C=low fluorescence, D=trace fluorescence and E=no detectable fluorescence The following results were obtained:

| | | | Fluorescense |
|---|---|---|---|
| Composition | Wash condition | Day light | with UV light |
| Guar gum- | Wash 1: Without | Bright yellow stain | A |
| DTAF | enzyme | left on textile | |
| Guar gum- | Wash 2: with | Faint yellow stain | C |
| DTAF | enzyme | left on textile | |

The results show that the composition left on the swatch after wash without enzyme was bright yellow in daylight and in UV light, the composition showed strong fluorescence. The composition left on the swatch after wash with enzyme showed a faint yellow stain in daylight and a low level of fluorescence in UV light.

### Example 14

Preparing and Testing a Composition (Pectin) Labelled with a Fluorophore (MIA)

100 mg pectin from citrus fruits (supplied by Sigma) was suspended in 20 mL of borate buffer (100 mM, pH8), heated to 100° C. while stirring for 30 minutes. Cooled down to room temperature and diluted with 20 ml of DMSO. Cooled to room temperature, and added 2 mL of freshly prepared MIA/DMSO solution (10 mg/ml). Stirred at room temperature for 60 minutes. Added 2 equivalents of ethanol, centrifugation (8500 rpm, 10 minutes), removal of the supernatant. Re-dissolved the solid residue in 40 mL of water, and heated to 100° C. for 20 minutes. Cooled to room temperature, and added 2 equivalents of ethanol, centrifugation (8500 rpm, 10 minutes), and removal of the supernatant. The solid residue was freeze-dried (Coolsafe CS110-4 pro freeze dryer (Scanvac)) overnight.

50 mg labelled galactomannan was dissolved in 2 ml deionized water, the solution (2.5%) was adjusted to pH 8, heated to 100° C. for 20 minutes, and applied 20 μl to surfaces on textile, dishware or hard surfaces.

Testing a Composition (Pectin) Labelled with a Fluorophore

The compositions of labelled galactomannan was prepared and 200 μl was applied to a textile swatch (Cotton 100% of WFK-10A)

The swatches were then washed according to Assay III according to the following table:

| Wash 1 | Wash 2 |
|---|---|
| Wash liquor comprising | Wash liquor comprising model detergent A |
| model detergent A 3.3 g/L | 3.3 g/L + 0.4 mg enzyme protein/L of |
| | pectate lyase SEQ ID NO: 3. |

### Evaluation and Results

The fluorescence labeled pectin stains were measured by using a Digieye as described in Assay I.

Scale from A-E, A=Strong fluorescence, B=medium fluorescence, C=low fluorescence, D=trace fluorescence and E=no detectable fluorescence The following results were obtained:

| | | | Fluorescense |
|---|---|---|---|
| Composition | Wash condition | Day light | with UV light |
| Pectin-MIA | Wash 1: Without | Not visible | E |
| | enzyme | composition left | |
| Pectin-MIA | Wash 2: with | Not visible | E |
| | enzyme | composition left | |

The results show that the composition left on the swatch was not visible after wash in visible light and UV light.

### Example 15

Preparing and Testing a Composition (Protein) Labelled with a Fluorophore (TRITC)

25 mg protein from Bovine serum albumin (supplied by Sigma) was suspended in 6 mL carbonate buffer (100 mM, pH9.5), and stirred until the solution became clear. Added 1.5 mL of freshly prepared TRITC/carbonate buffer solution (0.033 mg/ml) and stirred at room temperature for 120 minutes. The labelled protein was in a Nap25 columns (from Illustra). The column was prepared by washing in 2 times the column volumn using sodium phosphate buffer (20 mM, pH 7) and eluted by gravity. The reaction mixture was added to the column and eluted by gravity. The protein absorbed on the column was eluted with phosphate buffer. The first red band, containing TRITC labelled protein was collected. The next red band, containing unreacted TRITC, was discarded. The purification was repeated twice and the fractions containing TRITC labelled protein were pooled. The sample was evaporated with a SpeedVac (Sevant) Concentrator to 5 mg/mL. Labelled protein was added to a 10% whey protein solution and diluted in water to a final concentration of 0.8 mg/mL TRITC-protein and 5% whey solution, heated to 80° C. for 20 minutes, and applied 20 μl to surfaces on textile.

Testing a Composition (Protein) Labelled with a Fluorophore

The compositions of labelled protein were prepared and 20 μl was applied to a textile swatch (Cotton 100% of WFK-10A)

The swatches were then washed according to Assay III according to the following table:

| Wash 1 | Wash 2 |
|---|---|
| Wash liquor comprising | Wash liquor comprising model detergent A |
| model detergent A 3.3 g/L | 3.3 g/L + 4 mg enzyme protein/L of |
| | protease SEQ ID NO: 4 |

### Evaluation and Results

The fluorescence labeled rice starch stains were measured by using a Digieye as described in Assay I.

Scale from A-E, A=Strong fluorescence, B=medium fluorescence, C=low fluorescence, D=trace fluorescence and E=no detectable fluorescence The following results were obtained:

| | | | Fluorescense |
|---|---|---|---|
| Composition | Wash condition | Day light | with UV light |
| Protein- TRITC | Wash 1: Without | Pink stain left | A |
| | enzyme | on textile | |
| Protein- TRITC | Wash 2: with | No visible | E |
| | enzyme | composition left | |

The results show that the composition left on the swatch was visible in day light as a pink stain with no enzyme. Under UV light, the stain showed strong red fluorescence after was without enzyme. With enzyme, no composition was left on the swatch in either daylight or UV light.

1-15. (canceled)

16. An assay for testing the presence of a fluorophore on a first and/or a second surface comprising the following steps:
a. preparing a composition comprising a fluorophore having an absorbance wavelength and an emission wavelength,
b. applying the composition to the first surface,
c. submitting the first surface and/or the second surface to a liquid composition,
d. optionally rinsing and/or drying the first surface and/or the second surface,
e. exposing the surface and/or the second surface to light having a wavelength within +/−30 nm of the absorbance wavelength of the fluorophore, and
f. detecting the light emitted from the fluorophore present on the first surface and/or the second surface,
wherein the first and the second surface is present on a textile.

17. The assay of claim 16, wherein the fluorophore has an absorbance wavelength in the range of 200-810 nm and an emission wavelength in the range of 200-810 nm.

18. The assay of claim 16, wherein the fluorophore is selected from the group consisting of
a) Xanthene derivatives,
b) Cyanine derivatives,
c) Naphthalene derivatives,
d) Coumarin derivatives,
e) Oxadiazole derivatives,
f) Anthracene derivatives,
g) Pyrene derivatives,
h) Oxazine derivatives,
i) Acridine derivatives,
j) Arylmethine derivatives,
k) Tetrapyrrole derivatives,
I) Anthranilic acid derivatives, and
m) DTAF or NBD.

19. The assay of claim 16, wherein the composition comprises a raw material, a food product, material secreted from human or animal body, particulate material and/or a mixture thereof.

20. The assay of claim 19, wherein the composition comprises more than group of flourophore.

21. The assay of claim 16, wherein the liquid composition comprises a laundry detergent composition.

22. The assay of claim 16, wherein the liquid composition further comprises at least one enzyme.

23. A method for testing the presence of a fluorophore on a first and/or a second surface comprising the following steps:
a. preparing a composition comprising a fluorophore having an absorbance wavelength and an emission wavelength,
b. applying the composition to the first surface,
c. submitting the first surface and/or the second surface to a liquid composition,
d. optionally rinsing and/or drying the first surface and/or the second surface,
e. exposing the surface and/or the second surface to light having a wavelength within +/−30 nm of the absorbance wavelength of the fluorophore, and
f. detecting the light emitted from the fluorophore present on the first surface and/or the second surface,
wherein the first and the second surface is present on a textile.

24. The method of claim 23, wherein the fluorophore has an absorbance wavelength in the range of 200-810 nm and an emission wavelength in the range of 200-810 nm.

25. The method of claim 23, wherein the fluorophore is selected from the group consisting of
a. Xanthene
b. Cyanine derivatives,
c. Naphthalene derivatives,
d. Coumarin derivatives,
e. Oxadiazole derivatives,
f. Anthracene derivatives,
g. Pyrene derivatives,
h. Oxazine derivatives,
i. Acridine derivatives,
j. Arylmethine derivatives,
k. Tetrapyrrole derivatives,
I. Anthranilic acid derivatives, and
m. DTAF or NBD.

26. The method of claim 23, wherein the composition comprises a raw material, a food product, material secreted from human or animal body, particulate material and/or a mixture thereof.

27. The method of claim 23, wherein the liquid composition comprises a laundry detergent composition.

28. The method of claim 23, wherein the liquid composition further comprises at least one enzyme.

29. The method of claim 28, wherein the enzyme is selected from the group consisting of hemicellulases, peroxidases, proteases, cellulases, xylanases, lipases, phospholipases, esterases, cutinases, pectinases, mannanases, pectate lyases, keratinases, reductases, oxidases, phenoloxidases, lipoxygenases, ligninases, pullulanases, tannases, pentosanases, malanases, β-glucanases, arabinosidases, hyaluronidase, chondroitinase, laccase, DNase, chlorophyllases, amylases, perhydrolases, peroxidases, xanthanase and mixtures thereof.

30. A textile having a surface, which surface comprises a composition comprising a fluorophore.
